(19) United States
(12) Patent Application Publication
(10) Pub. No.: US 2023/0159457 A1
(43) Pub. Date: May 25, 2023

(54) FLUORENE DERIVATIVES, POLYMERS OBTAINED FROM SAID FLUORENE DERIVATIVES AND METHOD FOR PREPARING THE SAME

(71) Applicant: IRPC PUBLIC COMPANY LIMITED, Rayong (TH)

(72) Inventors: Roman HELMUTH ADAM STRAUSS, Rayong (TH); Banjong BUHIRAN, Rayong (TH); Sanya PRANGSRI-AROON, Krabi (TH); Jirarat KAMONSAWAS, Bang Khae, Bangkok (TH); Pranee LOOKSAARD, Rayong (TH); Ekkapon DAENGSOPHA, Ubon Ratchathani (TH); Vinich PROMARAK, Rayong (TH); Patteera FUNCHIEN, Trang (TH)

(73) Assignee: IRPC PUBLIC COMPANY LIMITED, Rayong (TH)

(21) Appl. No.: 17/919,333

(22) PCT Filed: Apr. 17, 2020
(86) PCT No.: PCT/TH2020/000024

§ 371 Date: Oct. 17, 2022

(51) Int. Cl. C07D 209/86; C07D 409/14; H10K 85/10; C09K 11/06; H10K 85/60; H10K 50/15

(52) U.S. Cl. CPC C07D 209/86; C07D 409/14; H10K 85/115; H10K 85/113; C09K 11/06; H10K 85/6576; H10K 85/6572; H10K 85/631; H10K 50/15; C09K 2211/1466; C09K 2211/1483

(57) ABSTRACT

The present invention relates to a novel fluorene derivative having a structure of formula (I): wherein X is independently selected from a group consisting of Cl, Br, I, trifluoromethanesulfonate and 4-(trifluoromethyl)benzenesulfonate; Y is a group of formula —(CH₂)ₙ— where n is an integer ranging from 3 to 12; and R¹, R², R³, and R⁴ are independently selected from a group consisting of tert-butyl group, triphenylamine, carbazole and carbazole derivative. The invention further relates to a polymer obtained from the said novel fluorene derivative and methods for preparing the same, as well as their uses as materials for an organic light-emitting diode device.

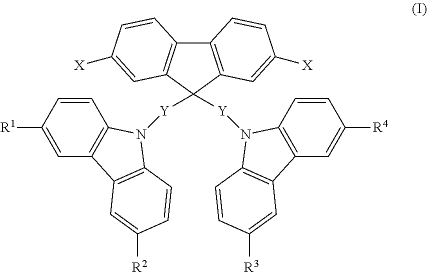

### TECHNICAL FIELD

The present invention relates to novel fluorene derivatives, polymers obtained from said fluorene derivatives and method for preparing the same, as well as their uses as materials for an organic light-emitting diode.

### BACKGROUND OF THE INVENTION

The organic light-emitting diode (OLED) has recently drawn attention due to an increase in demand for various applications such as flat panel displays, automotive headlamps, advertising signage, packaging and general lighting. These because of its advantages over other light sources that are lower energy consumption, large area, flexible, and natural white light.

Basically, the OLED transforms electrical energy into light by applying current to an OLED material. It has a structure in which an organic material layer is interposed between an anode and a cathode. The organic material layer includes a multi-layer comprising different materials, for example an organic light-emitting layer (EL), a hole transport layer (HTL), an electron transport layer (ETL). The organic light-emitting material is classified as blue, green, and red light-emitting materials according to emitted colors.

In order to implement excellent performance of OLED, materials constituting the organic material layer should be stable and have good efficiency. Many attempts have been made to improve performances of OLED, for example:

US 20060284140 A1 discloses mixtures and conjugated polymers which contain bridged carbazole structural units and structural units which emit light from the triplet state. The mixtures comprise at least one conjugated polymer, at least one bridged carbazole unit and at least one triplet emitter.

WO 2000/046321 discloses fluorene copolymers, polymer blends comprising such copolymers, and electronic devices (such as polymer light-emitting diodes) containing one or more films derived from these copolymers. The copolymer comprises monomeric units in combination with at least 10 percent of the monomeric units are fluorene moieties selected from 9-substituted fluorene moieties, 9,9-disubstituted fluorene moieties or combinations thereof, and at least 1 percent of the monomeric units comprising two non-fluorene moieties which are different from each other but which both comprise delocalized-electrons and are independently selected from moieties that have hole transporting properties and moieties that have electron transporting properties.

KR 781921 B1 discloses a carbazole derivative and an organic electroluminescent device using the said carbazole derivative. The invention provides a compound of specified formula comprising carbazole as a center of a chrysene derivative with at least 2 amino acid residues and a substituted thereof.

Further examples of prior art can be found for instance in Lin-Peng Yu et al: “Modification of a donor-acceptor photovoltaic polymer by integration of optoelectronic moieties into its side chains”, POLYMER, Vol. 59, pages 57-66 XP029197836, WO 00/46321 A1, CN 102 936 332 B, and WO 2004/113468 A1.

However, development of the organic material layer forming material for OLED has not been satisfactory and thus there is a need for a novel derivatives and polymers that are suitable for producing the organic material layer.

### SUMMARY OF THE INVENTION

In a first aspect, the present invention relates to a fluorene derivative having a structure of formula (I):

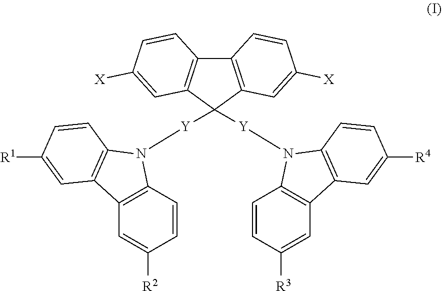

wherein

X is independently selected from a group consisting of Cl, Br, I, trifluoromethanesulfonate and 4-(trifluoromethyl)benzenesulfonate;

Y is a group of formula —(CH₂)ₙ— where n is an integer ranging from 3 to 12; and

R¹, R², R³ and R⁴ are independently selected from a group consisting of tert-butyl group, triphenylamine (TPA), carbazole and carbazole derivative.

In a second aspect, the present invention relates to a method for preparing a fluorene derivative having a structure of formula (I), the method comprising reacting a compound of formula (A) with a compound of formula (B) in an organic solvent in the presence of a strong base,

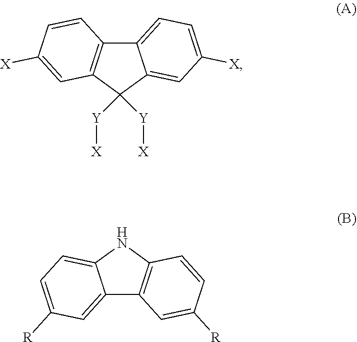

wherein

X is independently selected from a group consisting of Cl, Br, I, trifluoromethanesulfonate and 4-(trifluoromethyl)benzenesulfonate;

Y is a group of formula —(CH₂)ₙ— where n is an integer ranging from 3 to 12;

R¹, R², R³, R⁴ and R are independently selected from a group consisting of tert-butyl group, triphenylamine, carbazole and carbazole derivative; and

wherein a mole ratio of the compound of formula (A) to the compound of formula (B) to the strong base ((A):(B):strong base) is in a range of 1:2.5-3:8-9.

In a third aspect, the present invention relates to a polymer comprising a repeating unit derived from a fluorene derivative having a structure of formula (I).

In a fourth aspect, the present invention relates to a method for preparing the polymers that comprise a repeating unit derived from a fluorene derivative having a structure of formula (I), the method comprising a polymerization of monomers comprising fluorene derivatives having structures of formula (I) and (II)

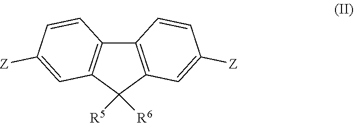

wherein

Z is dioxaborolane; and

R⁵ and R⁶ are independently selected from C₃-C₁₂ alkyl group.

In another aspect, the present invention relates to a use of polymers provided according to the invention as a hole transport layer in an electroluminescent device.

In further aspect, the present invention relates to a light-emitting element comprising the polymers provided according to the invention.

### DETAILED DESCRIPTION OF THE INVENTION

Unless otherwise indicated, any aspects shown herein shall encompass the application to other aspects of the present invention as well.

Unless otherwise specified, technical and scientific terms used herein have the definitions which are understood by a person skilled in the art.

Throughout the present invention, the term “about” is used to indicate that any values shown or presented herein may be varied or deviated. Such variation or deviation may be a result of an error of the equipment or method used to determine the values.

The terms “consist(s) of” and its variation such as “consisting of” and “consisted of”, “comprise(s)” and its variation such as “comprising” and “comprised”, “has/have/having”, “include(s)” and its variation such as “including” and “included” are open-ended verbs. For example, any methods which “consist of”, “comprise”, “have” or “include” one or more components or steps are not limited only to the one or more components or steps, but also cover the components or steps that are not mentioned.

The terms “a”, “an”, “the”, when used to refer to a singular noun, are intended to include a plural of that noun as well, unless otherwise specified.

Any tools, equipment, methods, materials or chemicals mentioned herein, unless otherwise indicated, mean the tool, equipment, methods, materials or chemicals generally used or practiced by a person skilled in the art.

### Fluorene Derivatives of the Invention

According to the first aspect, the present invention provides a fluorene derivative having a structure of formula (I):

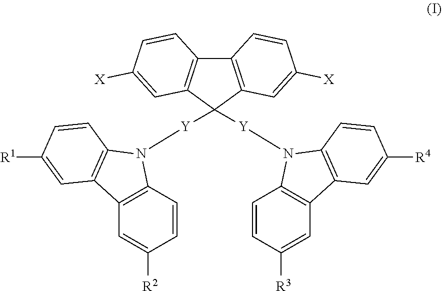

wherein

X is independently selected from a group consisting of Cl, Br, I, trifluoromethanesulfonate and 4-(trifluoromethyl)benzenesulfonate;

Y is a group of formula —(CH₂)ₙ— where n is an integer ranging from 3 to 12; and

R¹, R², R³ and R⁴ are independently selected from a group consisting of tert-butyl group, triphenylamine, carbazole and carbazole derivative.

In a preferred embodiment, X is Br or I. Y is a group of formula —(CH₂)ₙ— where n is an integer ranging from 3 to 10.

Preferably, carbazole derivative is tert-butyl-substituted carbazole.

More preferably, tert-butyl-substituted carbazole which can be used according to the present invention is selected from 3,6-di-tert-butylcarbazole and 3,3″,6,6″-tetra-tert-butyl-9′H-9,3′: 6′,9″-tercarbazole.

Examples of fluorene derivatives according to the present invention have the following structures:

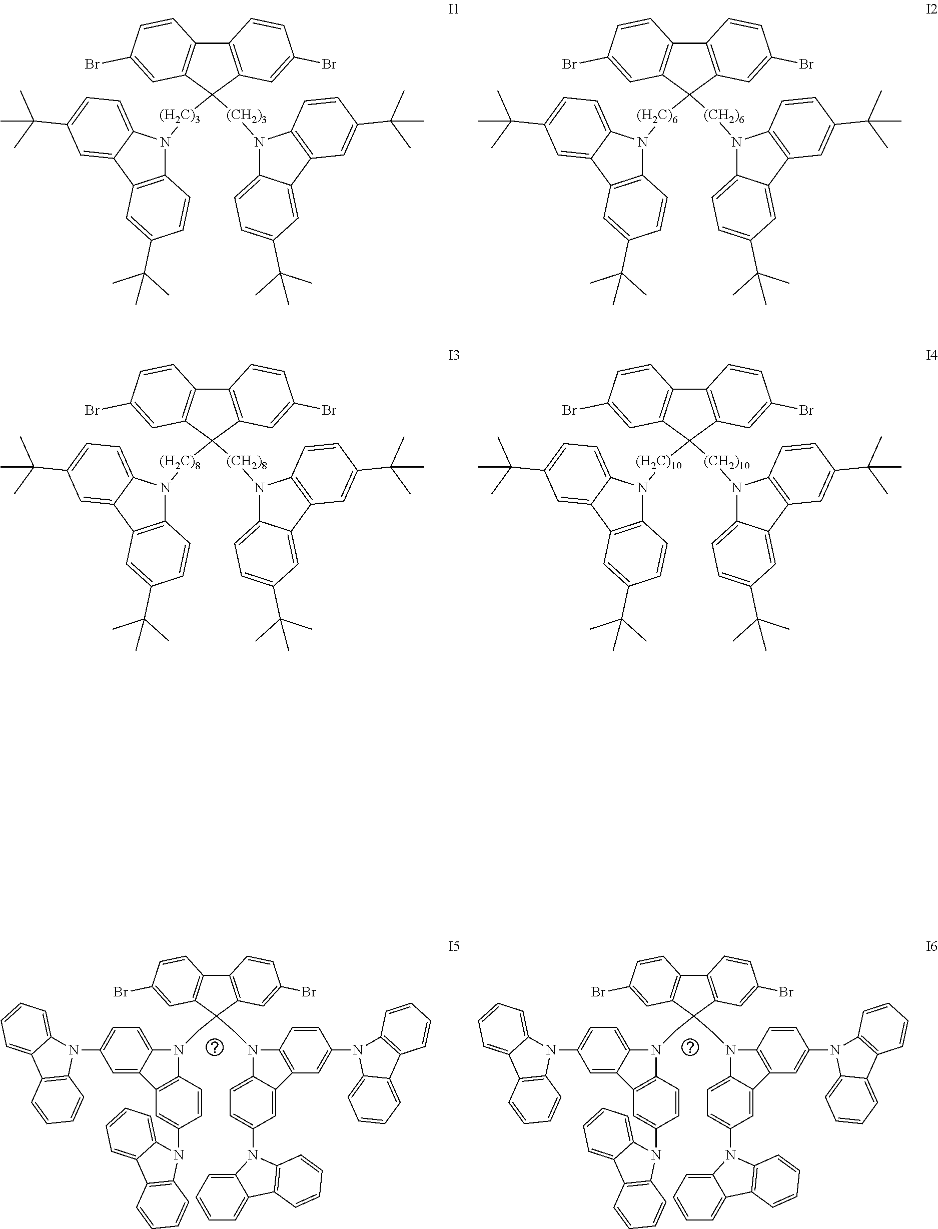

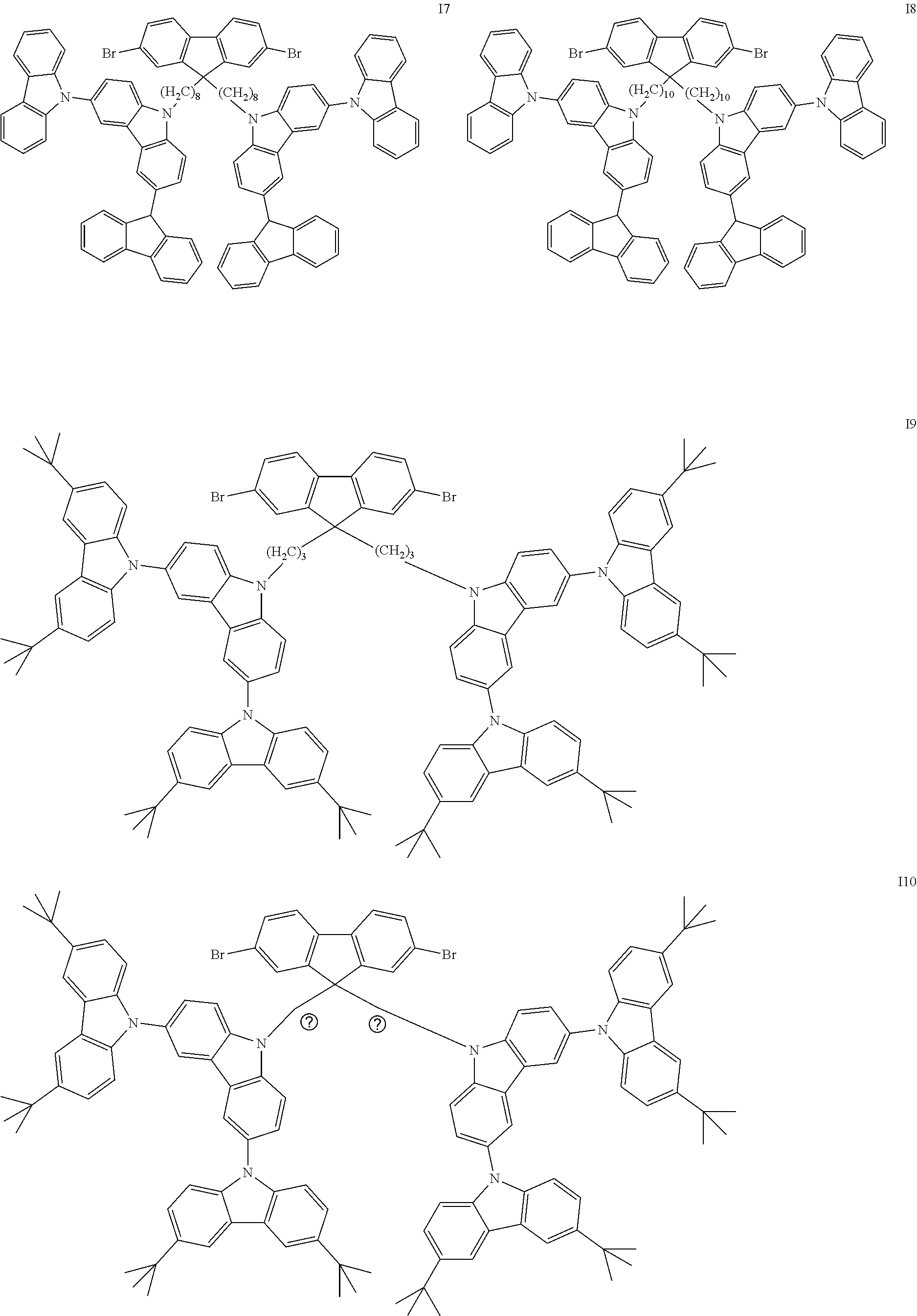

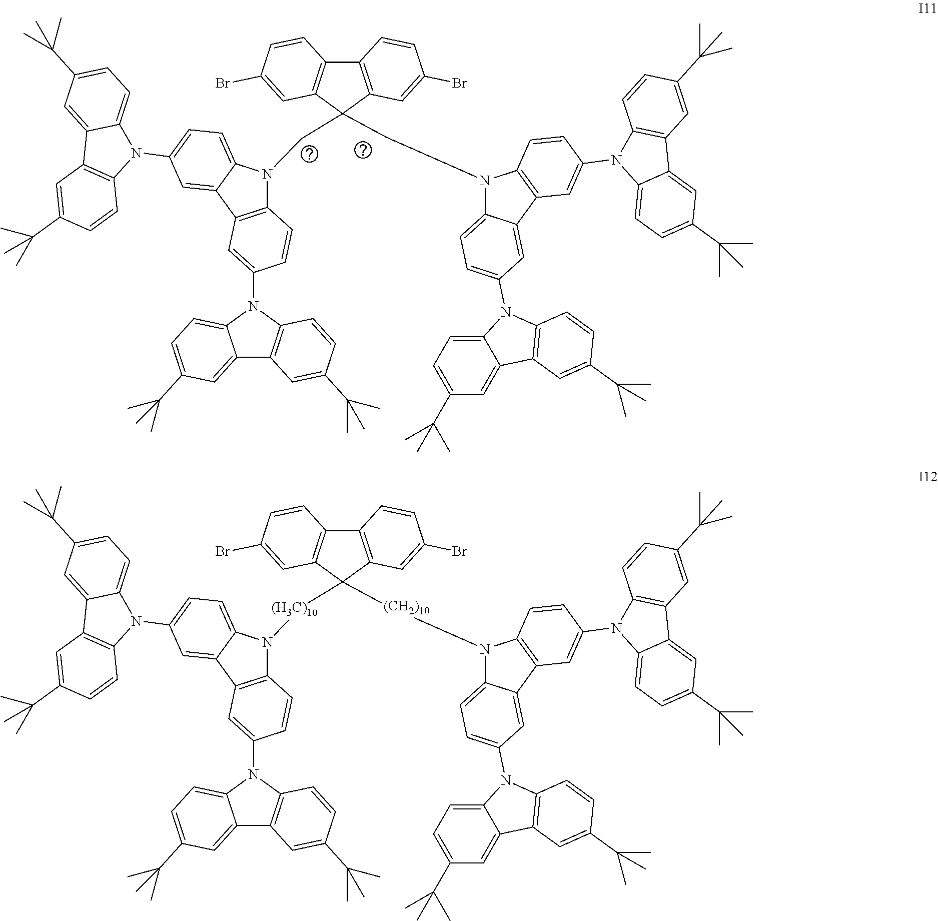

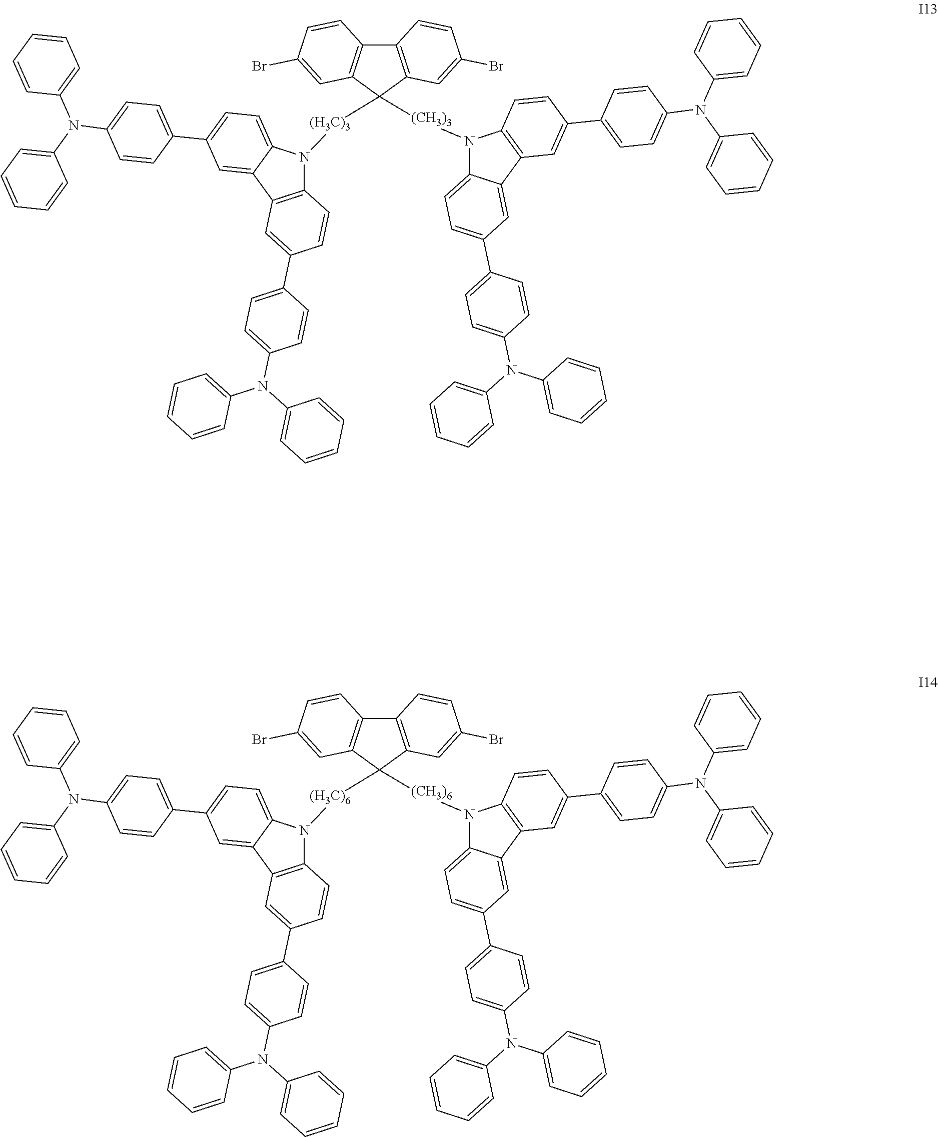

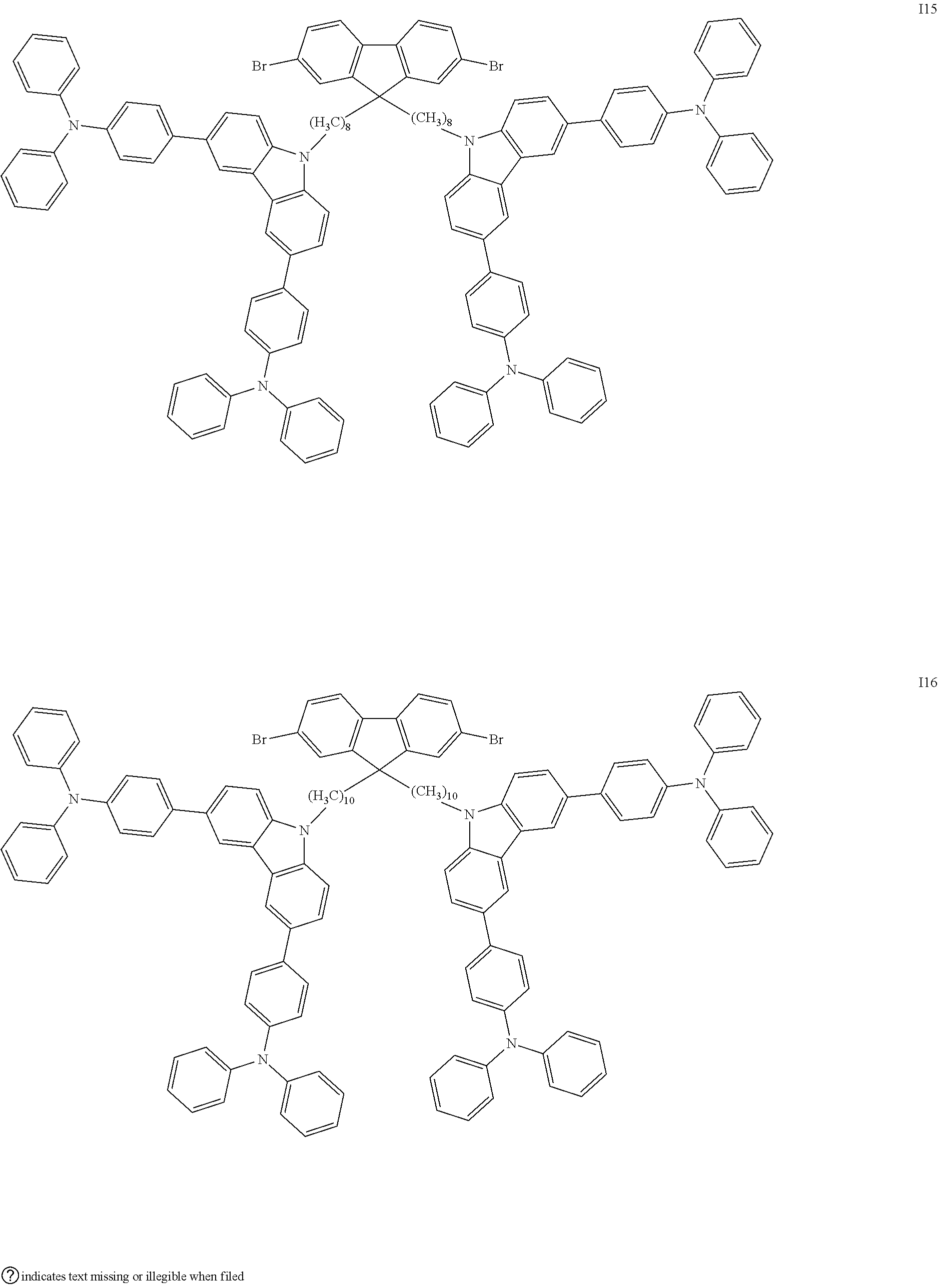

### Preparation of Fluorene Derivatives

According to the second aspect, the present invention also provides a method for preparing the fluorene derivative having the structure of formula (I), wherein the symbols X, Y, R¹, R², R³ and R⁴ have the same meanings as defined above. The method according to this invention comprises reacting a compound of formula (A) with a compound of formula (B) in an organic solvent in the presence of a strong base,

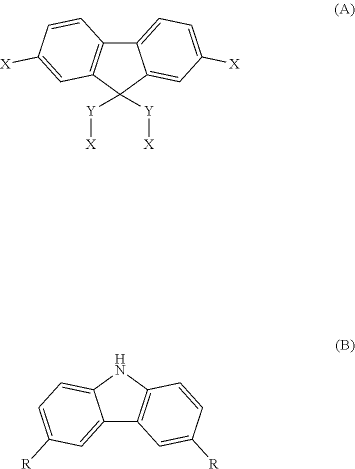

wherein

X is selected from a group consisting of Cl, Br, I, trifluoromethanesulfonate and 4-(trifluoromethyl)benzenesulfonate;

Y is a group of formula —(CH₂)ₙ— where n is an integer ranging from 3 to 12;

Each R is independently selected from a group consisting of tert-butyl group, triphenylamine, carbazole and carbazole derivative.

The preferred fluorene derivatives according to the present invention can be obtained by using an appropriate specific mole ratio and types of the reactants and reagents.

In a preferred embodiment, the mole ratio of the compound of formula (A) to the compound of formula (B) to the strong base ((A):(B):strong base) is in a range of 1:2.5-3:8-9.

The organic solvents for use in the reaction of the compound of formula (A) and the compound of formula (B) may be selected from a group consisting of dimethylformamide (DMF), hexane, toluene, dichloromethane and chloroform.

According to this invention, the compounds of formula (A) can be obtained by using a specific procedure as described below.

The compound of formula (A) is prepared from steps comprising:

(i) reacting a fluorene compound with a halogenating agent or triflating agent in an organic solvent in the presence of a catalyst; and

(ii) reacting the compound obtained from the step (i) with a C₃-C₁₂ alkyl halide in an organic solvent in the presence of the halogenating agent or triflating agent and a strong base.

In a preferred embodiment, the step (i) is conducted at an ambient temperature for 3-5 hours and the step (ii) is conducted at a temperature of 70-80° C. for 4-5 hours. Further, the step (i) is carried out by using a mole ratio of the fluorene compound to the halogenating agent or triflating agent to the catalyst (fluorene compound:halogenating agent or triflating agent:catalyst) is in a range of 1:2-3:0.05-0.1.

Preferably, the halogenating agent that can be used in the step (i) is selected from a group consisting of dibromine (Br₂) and tert-butyl ammonium bromide.

Preferably, the triflating agent that can be used in the step (i) is selected from a group consisting of trifluoromethanesulfonate and 4-(trifluoromethyl)benzenesulfonate.

The catalyst that can be used in step (i) is preferably selected from a group consisting of metal halide. For example, the metal halide catalyst is iron (III) chloride (FeCl₃).

To obtain the desired compound of formula (A), an appropriate specific mole ratio and types of the reactants and reagents are used. In a preferred embodiment, the step (ii) is carried out by using a mole ratio of the compound obtained from step (i) to the C₃-C₁₂ alkyl halide to the halogenating agent or triflating agent to the strong base (compound obtained from step (i): C₃-C₁₂ alkyl halide:halogenating agent or triflating agent:strong base) in a range of 1:3-5:0.1-0.3:8-15.

Preferably, the C₃-C₁₂ alkyl halide that can be used in step (ii) is selected from a group consisting of 1,3-dibromopropane, 1,6-dibromohexane, 1,8-dibromooctane and 1,10-dibromodecane.

Preferably, the organic solvents that can be used to perform the reaction of step (i) and (ii) may be commercially available and selected from a group consisting of chloroform, tetrahydrofuran (THF), dichloromethane, toluene, and hexane.

Examples of the compounds of formula (A) have structures as shown below.

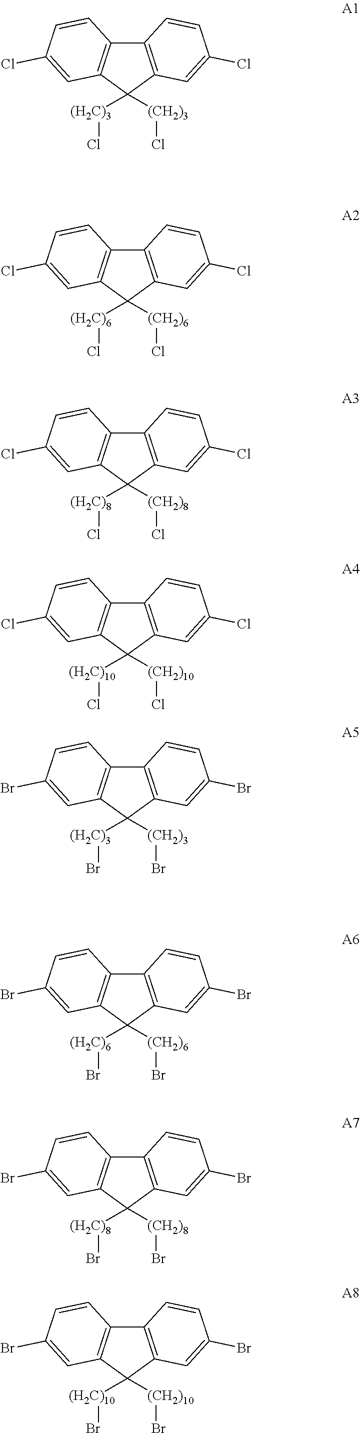

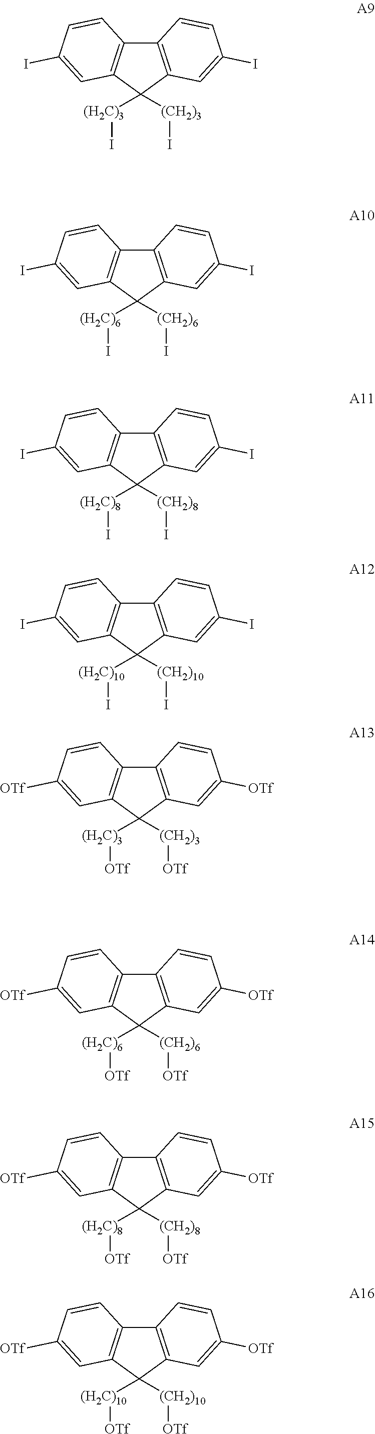

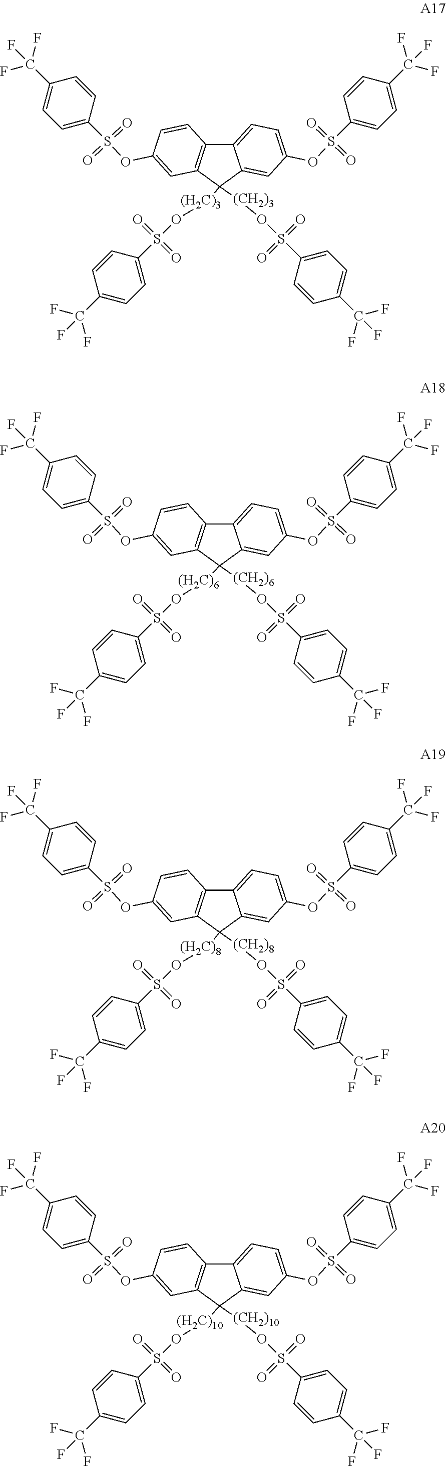

According to this invention, the compounds of formula (B) can be obtained by using a procedure as described herein below.

The compound of formula (B) is prepared by reacting carbazole with a halogenated compound or triflating agent in a mixed organic solvent in the presence of a catalyst.

The reaction between the carbazole and the halogenated compound or triflating agent can be achieved by using an appropriate specific mole ratio of the reactants and catalyst. In a preferred embodiment, the preparation of the compound of formula (B) is carried out by using a mole ratio of carbazole to the halogenated compound or triflating agent to the catalyst (carbazole:halogenating compound or triflating agent:catalyst) in a range of 1:2.5-3.5:2.5-3.5.

Preferably, the halogenated compound is 2-chloro-2-methylpropane, the triflating agent is selected from a group consisting of trifluoromethanesulfonate and 4-(trifluoromethyl) benzenesulfonate. The catalyst is selected from a group of metal halide such as zinc (II) chloride.

The mixed organic solvent suitable for use in the reaction is a mixture of two or more organic solvents in an appropriate volume ratio that can at least partially dissolve the carbazole, halogenated compound and catalyst used in this invention. Preferably, the organic solvents may be any commercially available and selected from a group consisting of nitromethane (CH₃NO₂), dichloromethane, hexane and toluene.

In an embodiment, the mixed organic solvent is the mixture of nitromethane and dichloromethane and a volume ratio of nitromethane and dichloromethane is 2 to 1.

According to this invention, the compound of formula (B) is prepared by steps of:

(i) reacting 3,6-dihalo-N-tosyl carbazole with carbazole or carbazole derivative in an organic solvent in the presence of a catalyst and a strong base; and

(ii) deprotecting a tosyl group of the compound obtained from step (i) in a mixed organic solvent in the presence of a strong base.

Preferably, the step (i) is conducted at a temperature of 110-120° C. under refluxing condition for 40-50 hours. The step (ii) is conducted at a temperature of 70-80° C. under refluxing condition for 3-5 hours.

In a preferred embodiment, the step (i) is carried out by using a mole ratio of 3,6-dihalo-N-tosyl carbazole to carbazole or carbazole derivative to the catalyst (3,6-dihalo-N-tosyl carbazole:carbazole or carbazole derivative:catalyst) in a range of 1:2.2-2.5:0.5-0.8. The 3,6-dihalo-N-tosyl carbazole is preferably 3,6-diiodo-9-tosyl-9H-carbazole.

The catalyst used in the reaction between 3,6-dihalo-N-tosyl carbazole and carbazole, or 3,6-dihalo-N-tosyl carbazole and carbazole derivative according to the step (i) may be in a form of a transition metal-ligand complex, or a transition metal precursor/ligand mixture. In one embodiment, the catalyst can be provided by mixing a transition metal precursor and a ligand compound in an appropriate organic solvent. Preferably, the catalyst can be selected from a group consisting of metal halide and diamine compound.

In a preferred embodiment, the catalyst of step (i) is prepared from copper(I) iodide (CuI) and trans-diaminocyclohexane.

The organic solvent that can be used in the preparation of the compound of formula (B) may be commercially available and selected from toluene, dichloromethane, hexane, dimethyl sulfoxide and THF. Preferably, the organic solvent of step (i) is toluene and the mixed organic solvent step (ii) is a mixture of tetrahydrofuran (THF) and dimethyl sulfoxide (DMSO).

Examples of the compounds of formula (B) have structures as shown below.

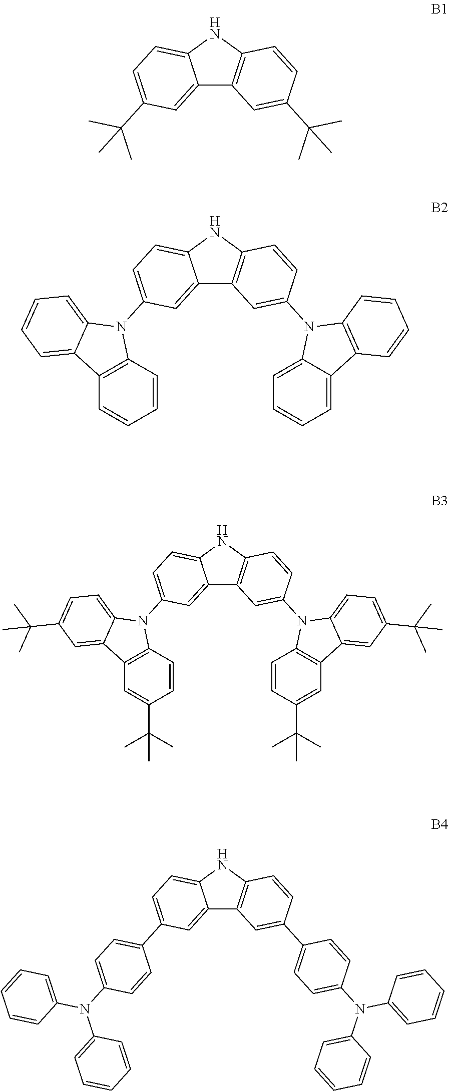 [SMILES: CC(C)(C)c1ccc2[nH]c3ccc(C(C)(C)C)cc3c2c1 ; c1ccc2c(c1)c1ccccc1n2-c1ccc2[nH]c3ccc(-n4c5ccccc5c5ccccc54)cc3c2c1 ; CC(C)(C)c1ccc2c(c1)c1cc(C(C)(C)C)ccc1n2-c1ccc2[nH]c3ccc(-n4c5ccc(C(C)(C)C)cc5c5cc(C(C)(C)C)ccc54)cc3c2c1 ; c1ccc(N(c2ccccc2)c2ccc(-c3ccc4[nH]c5ccc(-c6ccc(N(c7ccccc7)c7ccccc7)cc6)cc5c4c3)cc2)cc1]

### Polymers of the Invention

According to the third aspect, the present invention provides a polymer comprising a repeating unit derived from a fluorene derivative having a structure of formula (I), wherein the symbols X, Y, R¹, R², R³ and R⁴ have the same meanings as defined above.

In a preferred embodiment, X is Br or I. Y is a group of formula —(CH₂)ₙ— where n is an integer ranging from 3 to 10.

The carbazole derivative according to the present invention is preferably Cert-butyl-substituted carbazole.

The tert-butyl-substituted carbazole according to the present invention is preferably 3,6-di-tert-butylcarbazole.

According to specific embodiments of the present invention, the polymer is provided in which the fluorene derivative unit of the invention is incorporated into a backbone of the polymer as described in detail below.

### Blue Light-Emitting Polymer

An embodiment of a blue light-emitting polymer of the present invention comprises a repeating unit derived from fluorene derivatives of formulas (I) and (II):

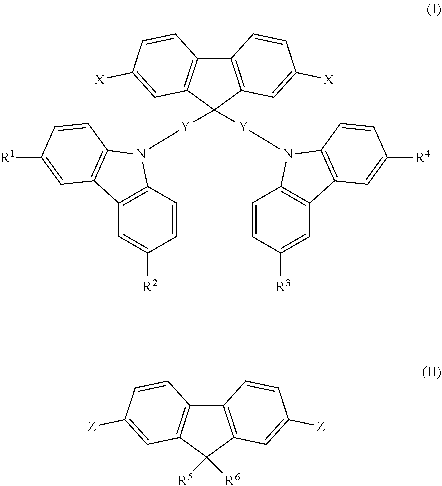

wherein

X is independently selected from a group consisting of Cl, Br, I, trifluoromethanesulfonate and 4-(trifluoromethyl)benzenesulfonate;

Y is a group of formula —(CH₂)ₙ— where n is an integer ranging from 3 to 12;

R¹, R², R³, and R^{4,} are independently selected from a group consisting of tert-butyl group, triphenylamine, carbazole and carbazole derivative;

Z is dioxaborolane; and

R⁵ and R⁶ are independently selected from C₃-C₁₂ alkyl group.

In a preferred embodiment, Y is a group of formula —(CH₂)ₙ— where n is an integer ranging from 3 to 10. R¹, R², R³ and R⁴ are identical. The carbazole derivative is tert-butyl-substituted carbazole. The preferred carbazole derivative can be selected from a group consisting of 3,6-di-tert-butylcarbazole and 3,3″,6,6″-tetra-tert-butyl-9′H-9,3′:6′,9″-tercarbazole. R⁵ and R⁶ are identical and are C₆-C₁₀ alkyl group.

The compounds having structures of formulas (I) and (II) which are used as monomers for preparing the blue light-emitting polymer according to the present invention can have any sequence of arrangement within the polymer chain.

Examples of the blue light-emitting polymers according to the present invention have structures as shown below.

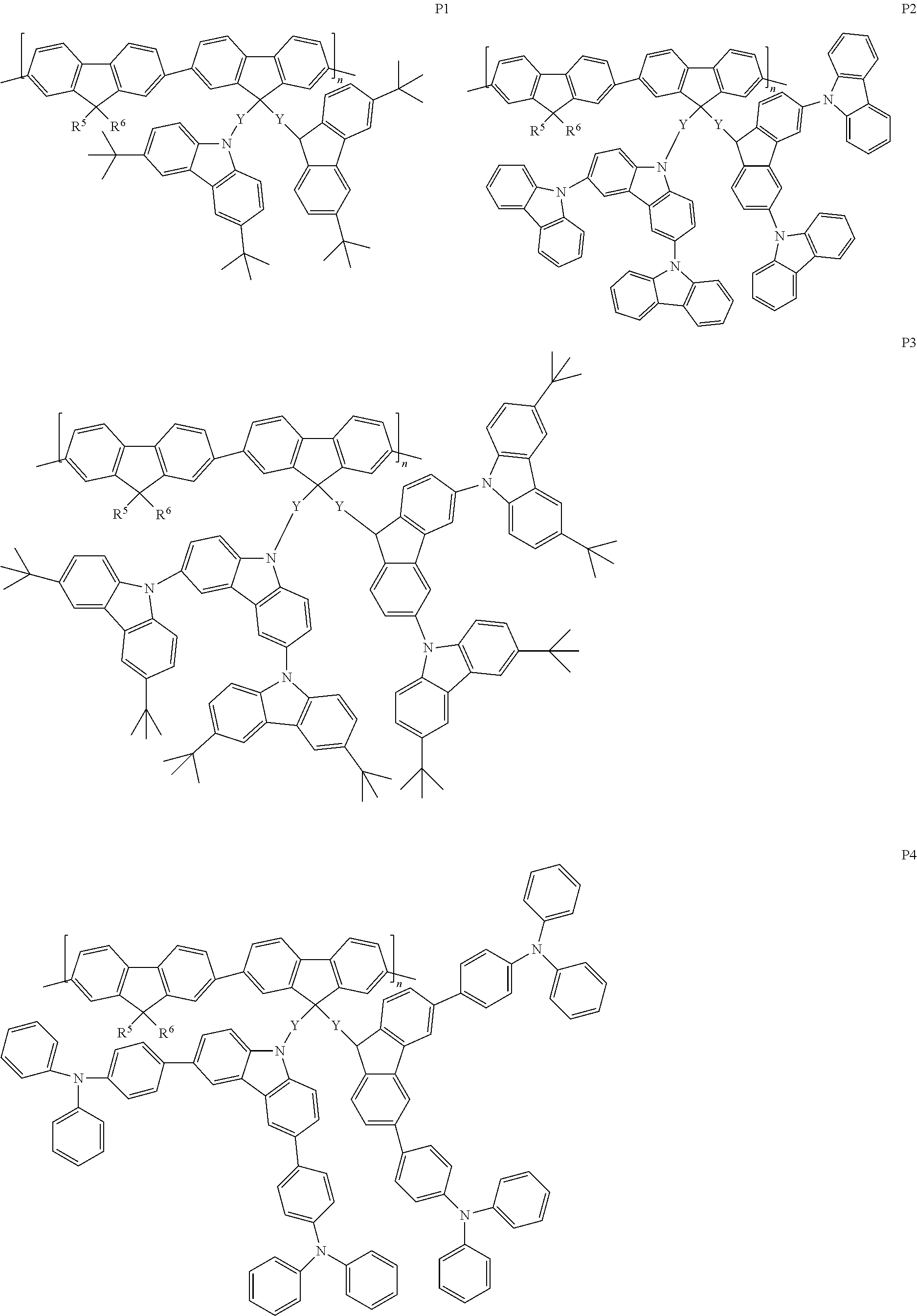

It is preferable that the blue light-emitting polymer according to the present invention has a weight average molecular weight (Mw) in a range of 6,000 to 60,000 g/mol, a number average molecular weight (Mn) in a range of 4,200 to 30,000 g/mol and a polydispersity index (PDI) in a range of 1 to 5.

In a preferred embodiment, the blue light-emitting polymer according the present invention has a mole ratio of formula (II) to formula (I) in a range of 1:0.3-1, preferably 1 to 1.

In one embodiment, the blue light-emitting polymer has a peak wavelength of light emitted in a visible spectrum of 350 nm to 385 nm.

In further embodiment, the blue light-emitting polymer has CIE coordinates of light emission of x=0.16 to 0.18, y=0.07 to 0.13.

It is preferable that the polymer is used as a blue electroluminescent material.

### Red Light-Emitting Polymer

An embodiment of a red light-emitting polymer of the present invention comprises a repeating unit derived from fluorene derivatives of formulas (I) and (II) and a compound of formula (III):

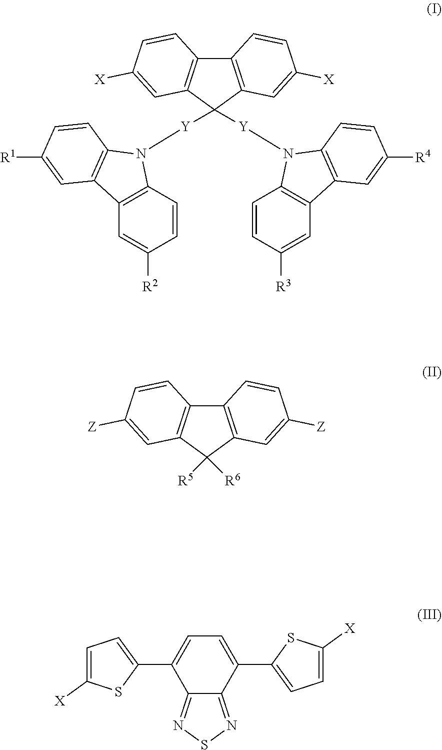

wherein

X is independently selected from a group consisting of Cl, Br, I, trifluoromethanesulfonate and 4-(trifluoromethyl)benzenesulfonate;

Y is a group of formula —(CH₂)ₙ— where n is an integer ranging from 3 to 12;

R¹, R², R³, and R^{4,} are independently selected from a group consisting of tert-butyl group, triphenylamine, carbazole and carbazole derivative;

Z is dioxaborolane; and

R⁵ and R⁶ are independently selected from C₃-C₁₂ alkyl group.

In a preferred embodiment, Y is a group of formula —(CH₂)ₙ— where n is an integer ranging from 3 to 10. R¹, R², R³ and R⁴ are identical. The carbazole derivative is tert-butyl-substituted carbazole. The preferred carbazole derivative can be selected from a group consisting of 3,6-di-tert-butylcarbazole and 3,3″,6,6″-tetra-tert-butyl-9′H-9,3′:6′,9″-tercarbazole. R⁵ and R⁶ are identical and are C₆-C₁₀ alkyl group.

The derivatives and compounds having structures of formulas (I), (II) and (III) which are used as monomers for preparing the red light-emitting polymer according to the present invention can have any sequence of arrangement within the polymer chain. For example, the monomer sequences for the red light-emitting polymer can be (I)-(II)-(III), (I)-(III)-(II), (II)-(I)-(III), (II)-(III)-(I), (III)-(I)-(II) or (III)-(II)-(I).

Examples of the red light-emitting polymers according to the present invention have structures as shown below.

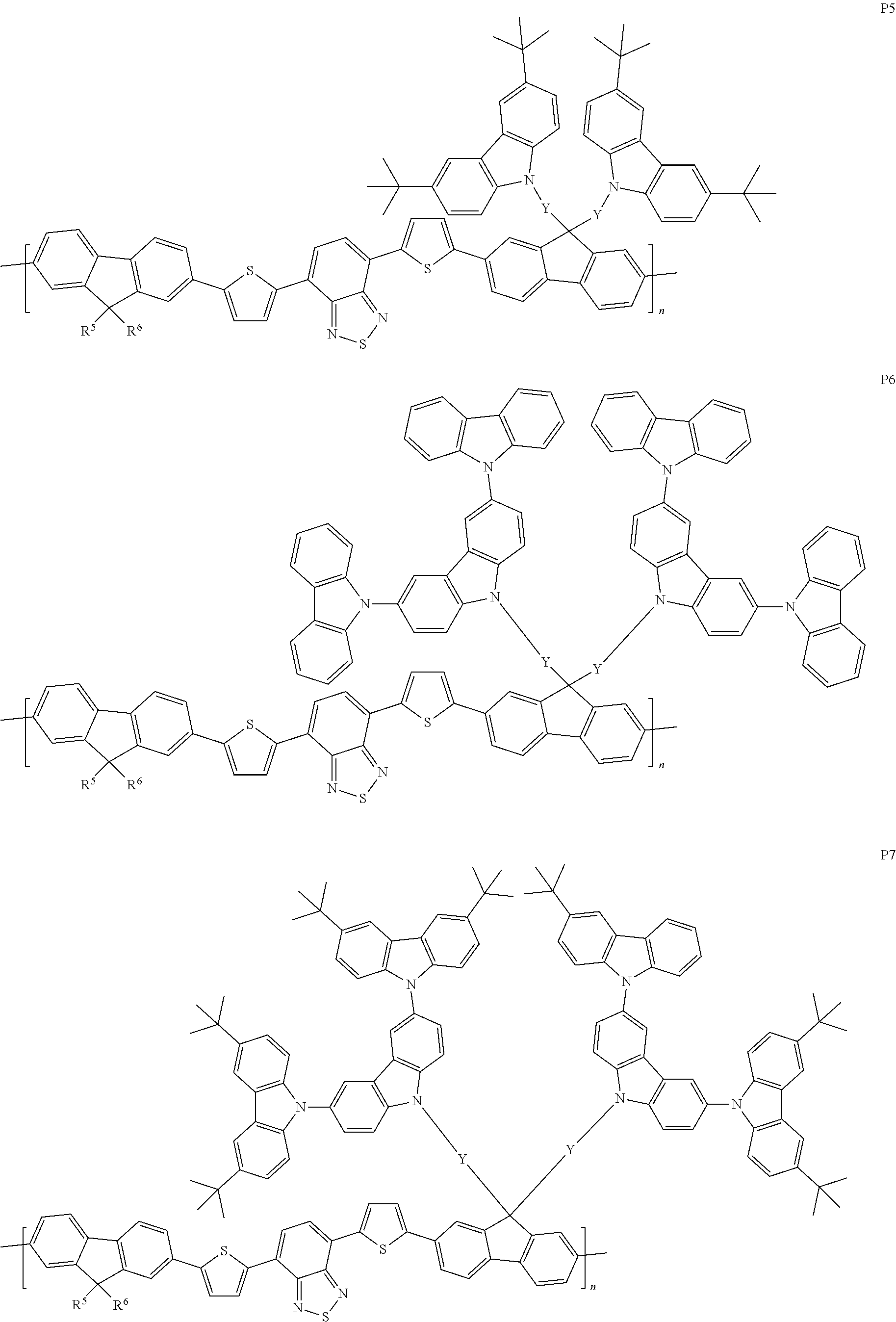

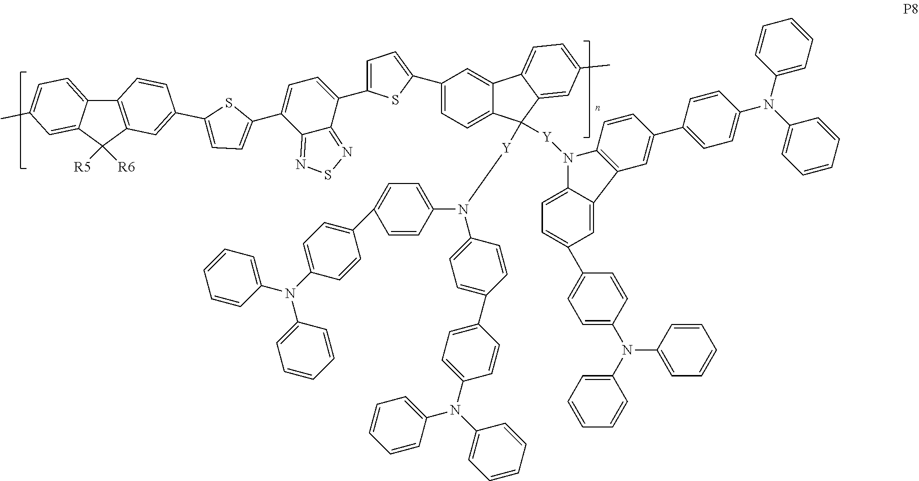

It is preferable that the red light-emitting polymer according to the present invention has a weight average molecular weight (Mw) in a range of 10,000 to 100,000 g/mol, a number average molecular weight (Mn) in a range of 10,000 to 35,000 g/mol and a polydispersity index (PDI) in a range of 1 to 5.

In a preferred embodiment, the red light-emitting polymer according to the present invention has a mole ratio of formula (II) to formula (I) to formula (III) in a range of 1:0.3-1:0.005-0.5.

In one embodiment, the red light-emitting polymer has a peak wavelength of light emitted in a visible spectrum of 370 nm to 390 nm and 540 nm to 560 nm.

In further embodiment, the red light-emitting polymer has CIE coordinates of light emission of x=0.68 to 0.72, y=0.28 to 0.31

It is preferable that the polymer is used as a red electroluminescent material.

### Green Light-Emitting Polymer

An embodiment of a green light-emitting polymer of the present invention comprises a repeating unit derived from fluorene derivatives of formulas (I) and (II) and a compound of formulas (IV) or (V):

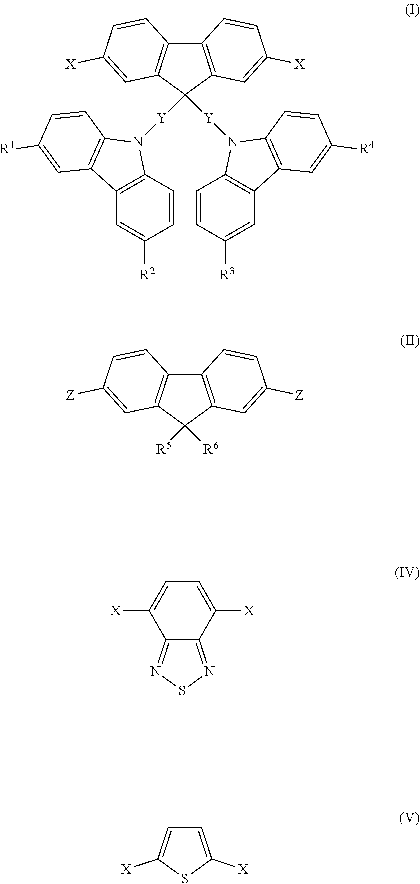

wherein

X is independently selected from a group consisting of Cl, Br, I, trifluoromethanesulfonate and 4-(trifluoromethyl)benzenesulfonate;

Y is a group of formula —(CH₂)ₙ— where n is an integer ranging from 3 to 12;

R¹, R², R³, and R^{4,} are independently selected from a group consisting of tert-butyl group, triphenylamine, carbazole and carbazole derivative;

Z is dioxaborolane; and

R⁵ and R⁶ are independently selected from C₃-C₁₂ alkyl group.

In a preferred embodiment, Y is a group of formula —(CH₂)ₙ— where n is an integer ranging from 3 to 10. R¹, R², R³ and R⁴ are identical. The carbazole derivative is tert-butyl-substituted carbazole. The preferred carbazole derivative can be selected from a group consisting of 3,6-di-tert-butylcarbazole and 3,3″,6,6″-tetra-tert-butyl-9′H-9,3′:6′,9″-tercarbazole. R⁵ and R⁶ are identical and are C₆-C₁₀ alkyl group.

The compounds having structures of formulas (I), (II), (IV) or (V) which are used as monomers for preparing the green light-emitting polymer according to the present invention can have any sequence of arrangement within the polymer chain. For example, the monomer sequences for the green light-emitting polymer can be (I)-(II)-(IV), (I)-(IV)-(II), (II)-(I)-(IV), (II)-(IV)-(I), (I)-(II)-(V), (I)-(V)-(II), (II)-(I)-(V) or (II)-(V)-(I).

Examples of the green light-emitting polymer according to the present invention have structures as shown below.

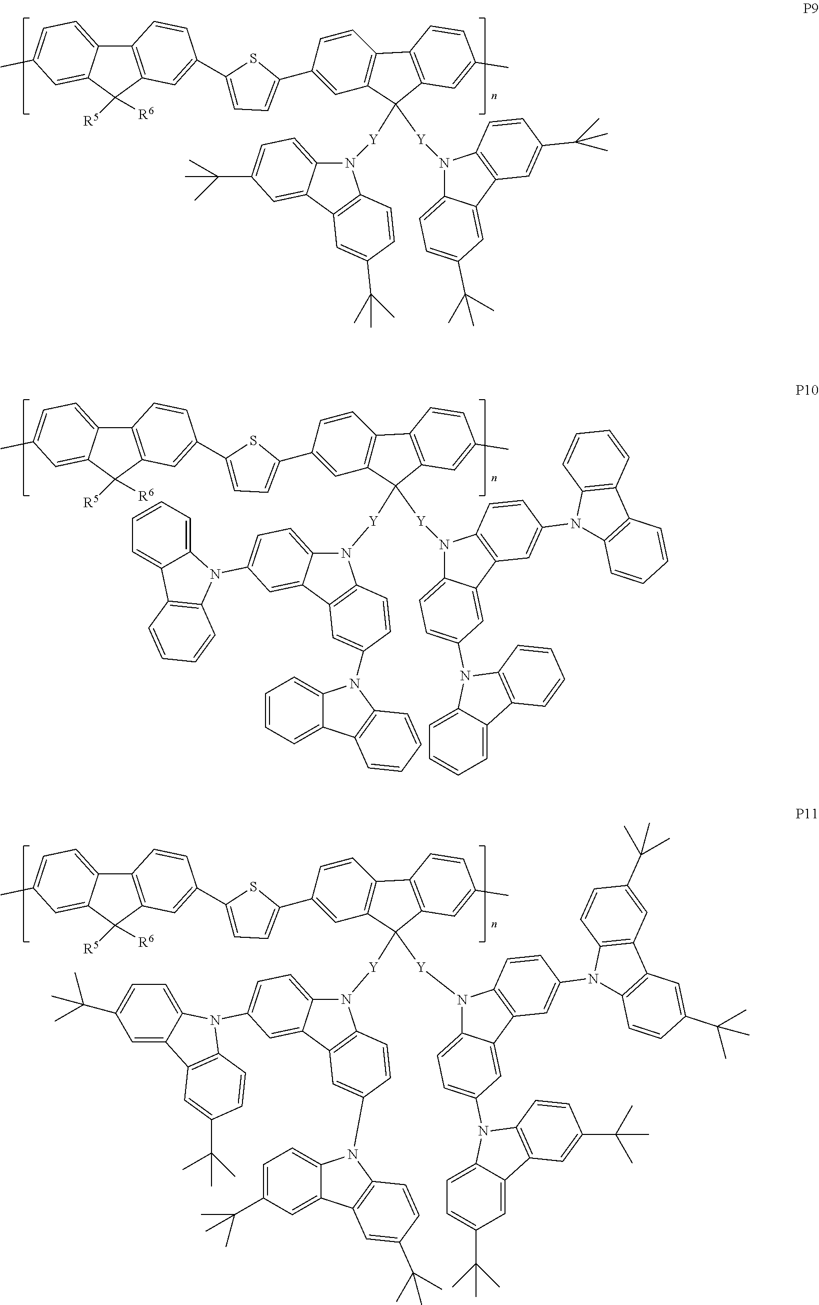

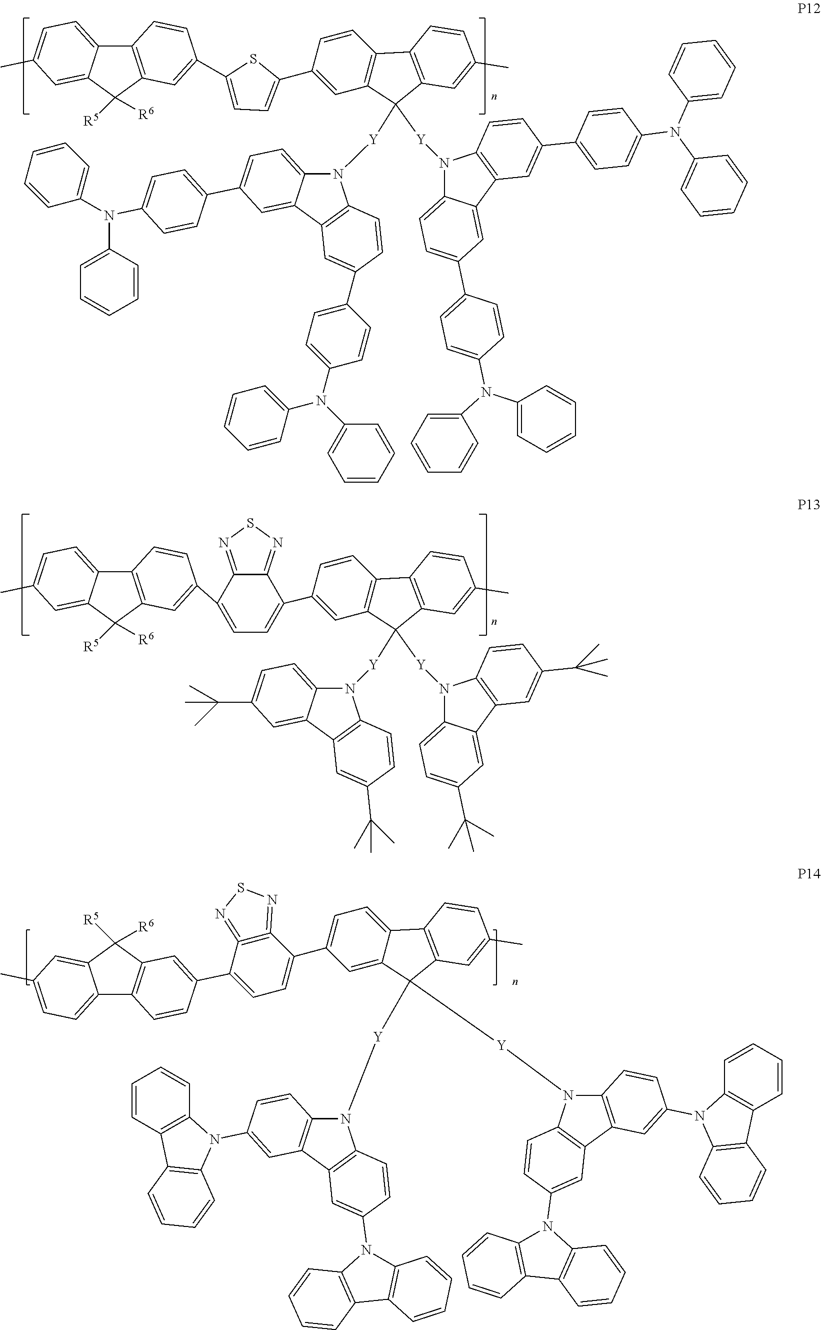

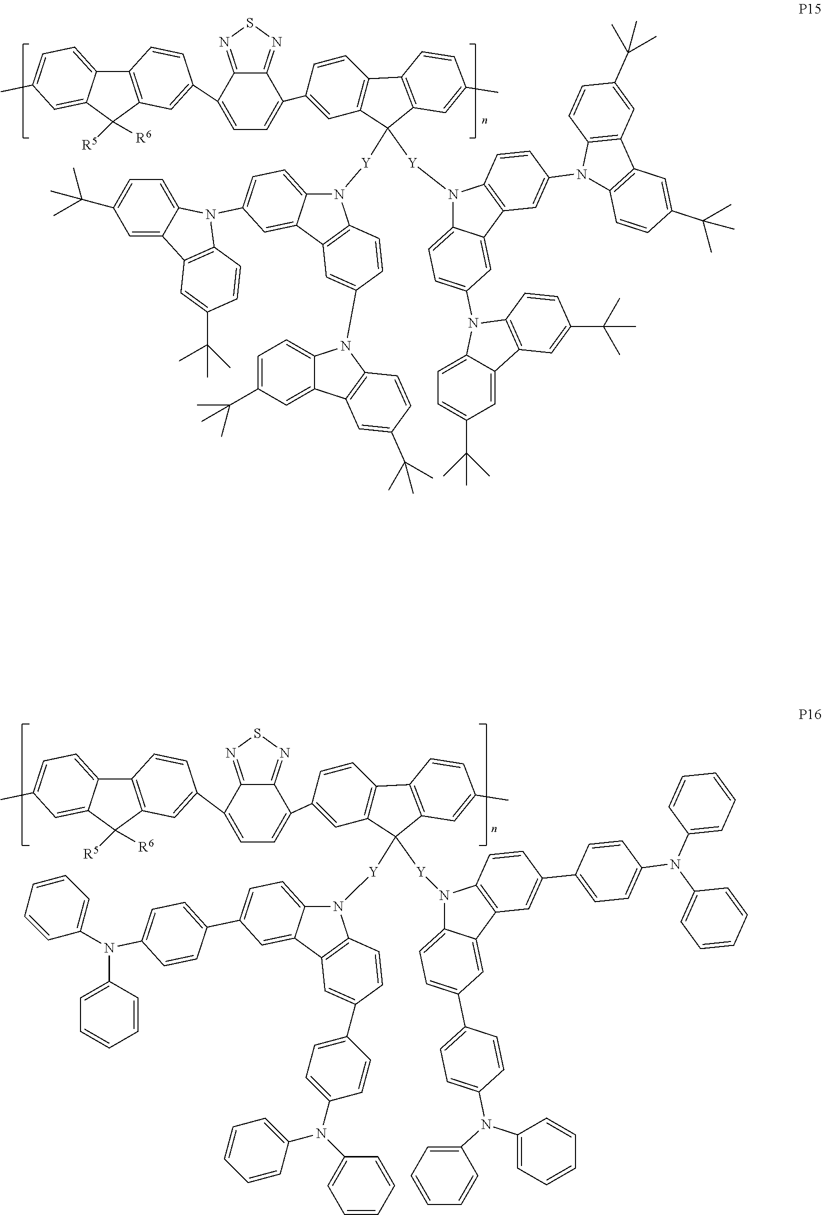

It is preferable that the green light-emitting polymer according to the present invention has a weight average molecular weight (Mw) in a range of 10,000 to 50,000 g/mol, a number average molecular weight (Mn) in a range of 10,000 to 30,000 g/mol and a polydispersity index (PDI) in a range of 1 to 5.

In a preferred embodiment, the green light-emitting polymer according to the present invention has a mole ratio of formula (II) to formula (I) to formula (IV) in a range of 1:0.3-1:0.008-0.7.

In a preferred embodiment, the green light-emitting polymer according to the present invention has a mole ratio of formula (II) to formula (I) to formula (V) in a range of 1:0.3-1:0.008-0.7.

In one embodiment, the green light-emitting polymer has a peak wavelength of light emitted in a visible spectrum of 430 nm to 440 nm.

In further embodiment, the green light-emitting polymer has CIE coordinates of light emission of x=0.32 to 0.36, y=0.52 to 0.60.

It is preferable that the polymer is used as a green electroluminescent material.

### White Light-Emitting Polymer

An embodiment of a white light-emitting polymer of the present invention comprises a repeating unit derived from fluorene derivatives of formulas (I) and (II) and a compound of formula (III) and a compound of formulas (IV) or (V):

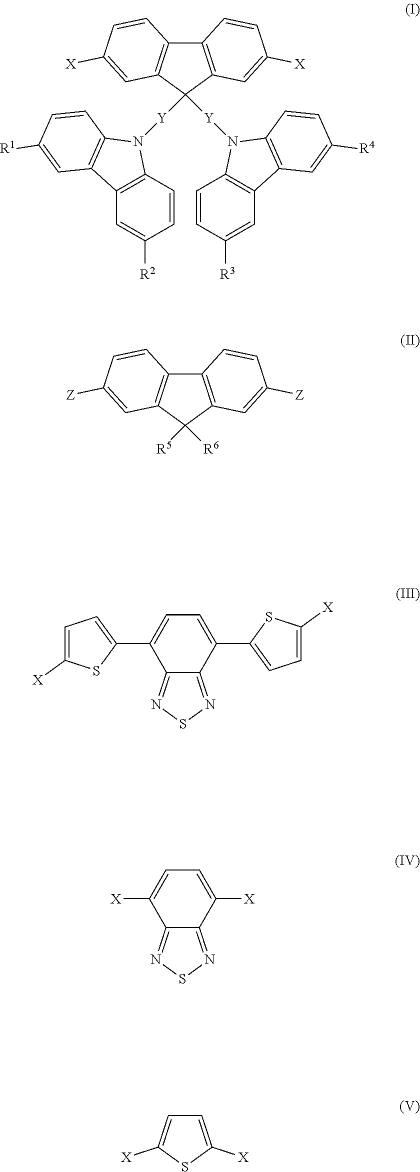

wherein

X is independently selected from a group consisting of Cl, Br, I, trifluoromethanesulfonate and 4-(trifluoromethyl)benzenesulfonate;

Y is a group of formula —(CH₂)ₙ— where n is an integer ranging from 3 to 12;

R¹, R², R³, and R^{4,} are independently selected from a group consisting of tert-butyl group, triphenylamine, carbazole and carbazole derivative;

Z is dioxaborolane; and

R⁵ and R⁶ are independently selected from C₃-C₁₂ alkyl group.

In a preferred embodiment, Y is a group of formula —(CH₂)ₙ— where n is an integer ranging from 3 to 10. R¹, R², R³ and R⁴ are identical. The carbazole derivative is tert-butyl-substituted carbazole. The preferred carbazole derivative can be selected from a group consisting of 3,6-di-tert-butylcarbazole and 3,3″,6,6″-tetra-tert-butyl-9′H-9,3′:6′,9″-tercarbazole. R⁵ and R⁶ are identical and are C₆-C₁₀ alkyl group.

The compounds having structures of formulas (I), (II), (III), (IV) or (V) which are used as monomers for preparing the white light-emitting polymer according to the present invention can have any sequence of arrangement within the polymer chain. For example, the monomer sequences for the white light-emitting polymer can be (I)-(II)-(III)-(IV), (I)-(II)-(IV)-(III), (I)-(III)-(IV)-(II), (I)-(III)-(II)-(IV), (I)-(IV)-(II)-(III), (I)-(IV)-(III)-(II), (II)-(I)-(III)-(IV), (II)-(I)-(IV)-(III), (II)-(III)-(IV)-(I), (II)-(III)-(I)-(IV), (II)-(IV)-(III)-(I), (II)-(IV)-(I)-(III), (III)-(I)-(II)-(IV), (III)-(I)-(IV)-(II), (III)-(II)-(I)-(IV), (III)-(II)-(IV)-(I), (III)-(IV)-(I)-(II), (III)-(IV)-(II)-(I), (IV)-(I)-(II)-(III), (IV)-(I)-(III)-(II), (IV)-(II)-(I)-(III), (IV)-(II)-(III)-(I), (IV)-(III)-(II)-(I), (IV)-(III)-(I)-(II), (I)-(II)-(III)-(V), (I)-(II)-(V)-(III), (I)-(III)-(V)-(II), (I)-(III)-(II)-(V), (I)-(V)-(II)-(III), (I)-(V)-(III)-(II), (II)-(I)-(III)-(V), (II)-(I)-(V)-(III), (II)-(III)-(V)-(I), (II)-(III)-(I)-(V), (II)-(V)-(III)-(I), (II)-(V)-(I)-(III), (III)-(I)-(II)-(V), (III)-(I)-(V)-(II), (III)-(II)-(I)-(V), (III)-(II)-(V)-(I), (III)-(V)-(I)-(II), (III)-(V)-(II)-(I), (V)-(I)-(II)-(III), (V)-(I)-(III)-(II), (V)-(II)-(I)-(III), (V)-(II)-(III)-(I), (V)-(III)-(II)-(I), (V)-(III)-(I)-(II).

Examples of the white light-emitting polymer according to the present invention have structures as shown below.

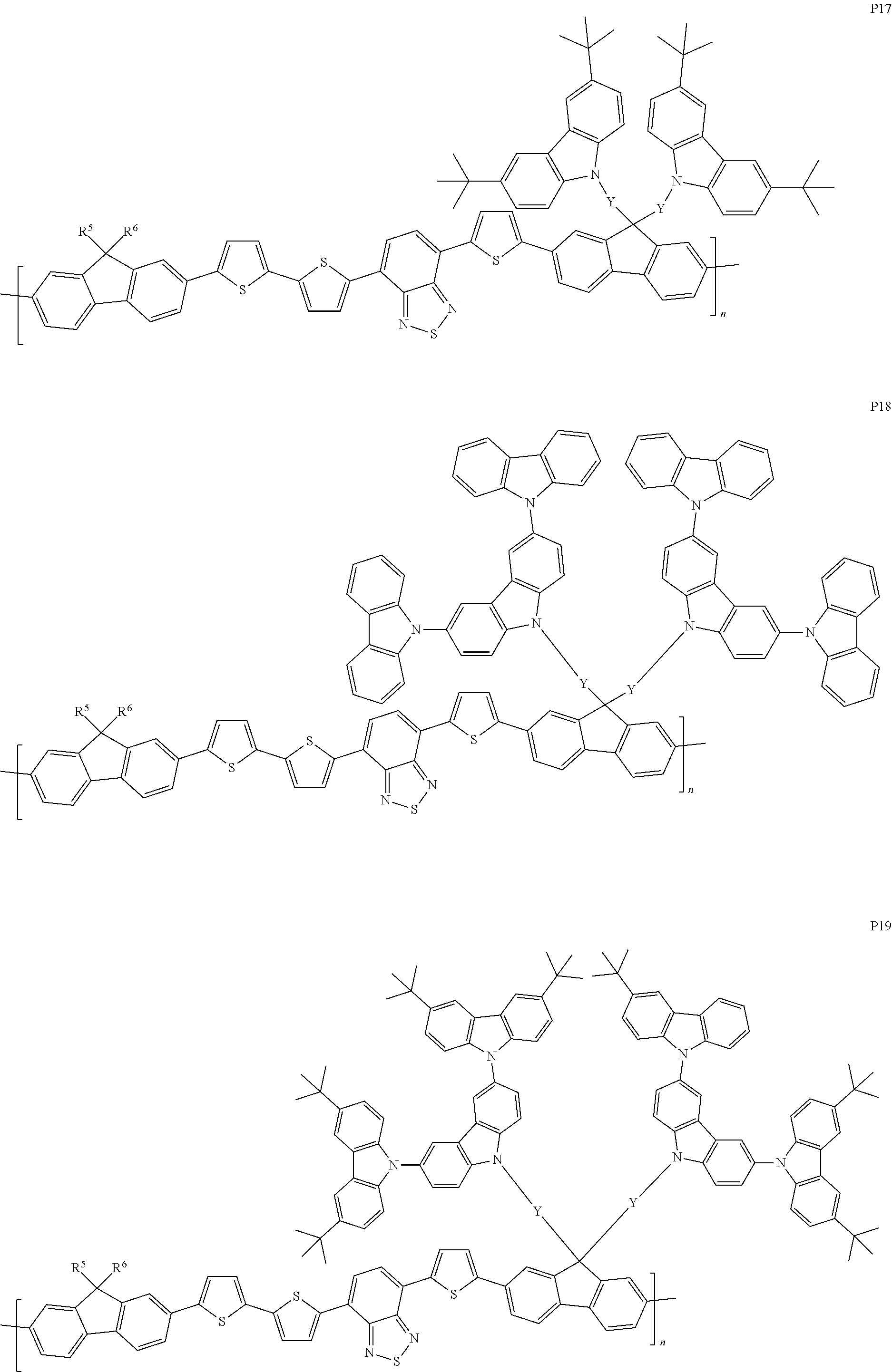

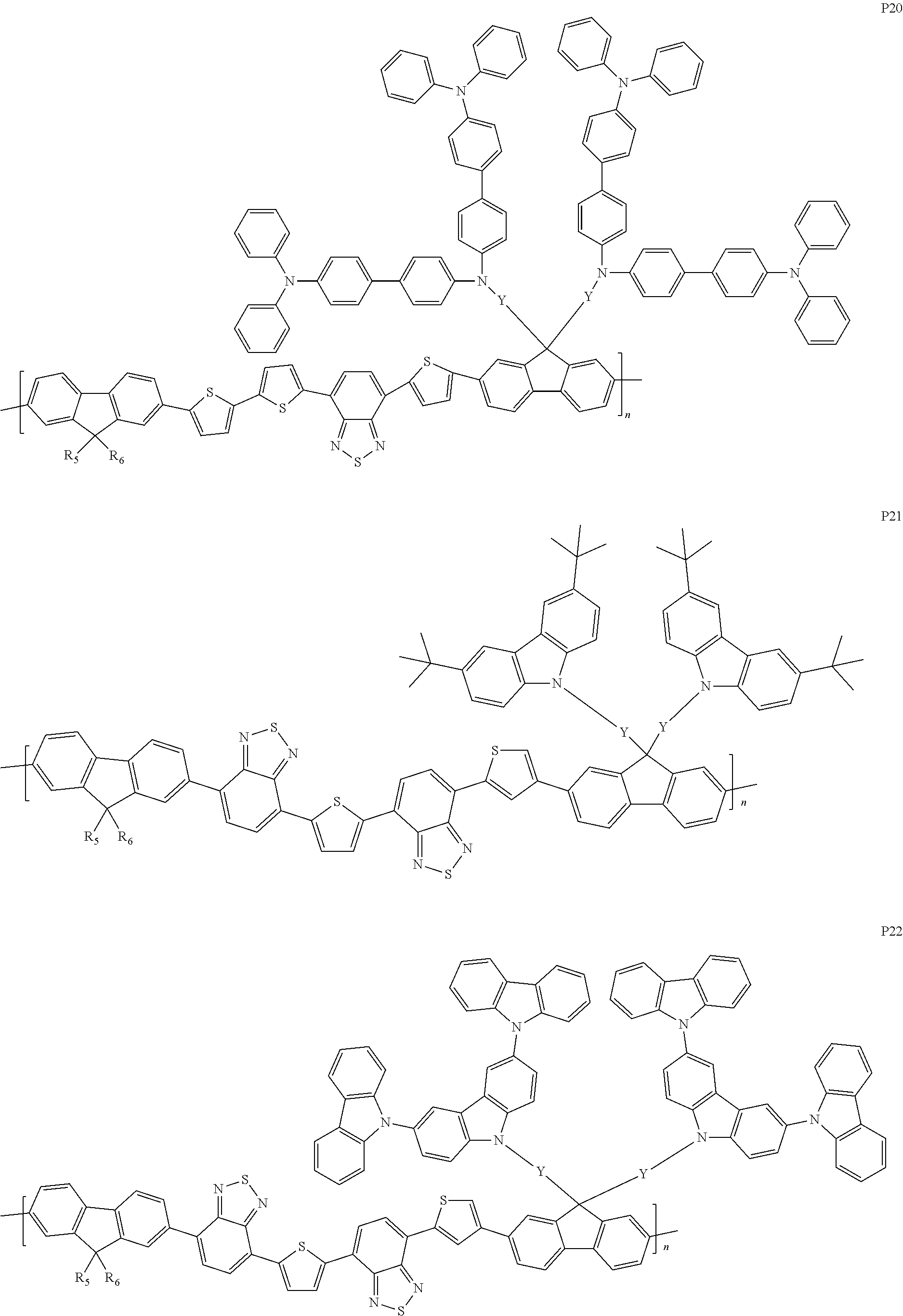

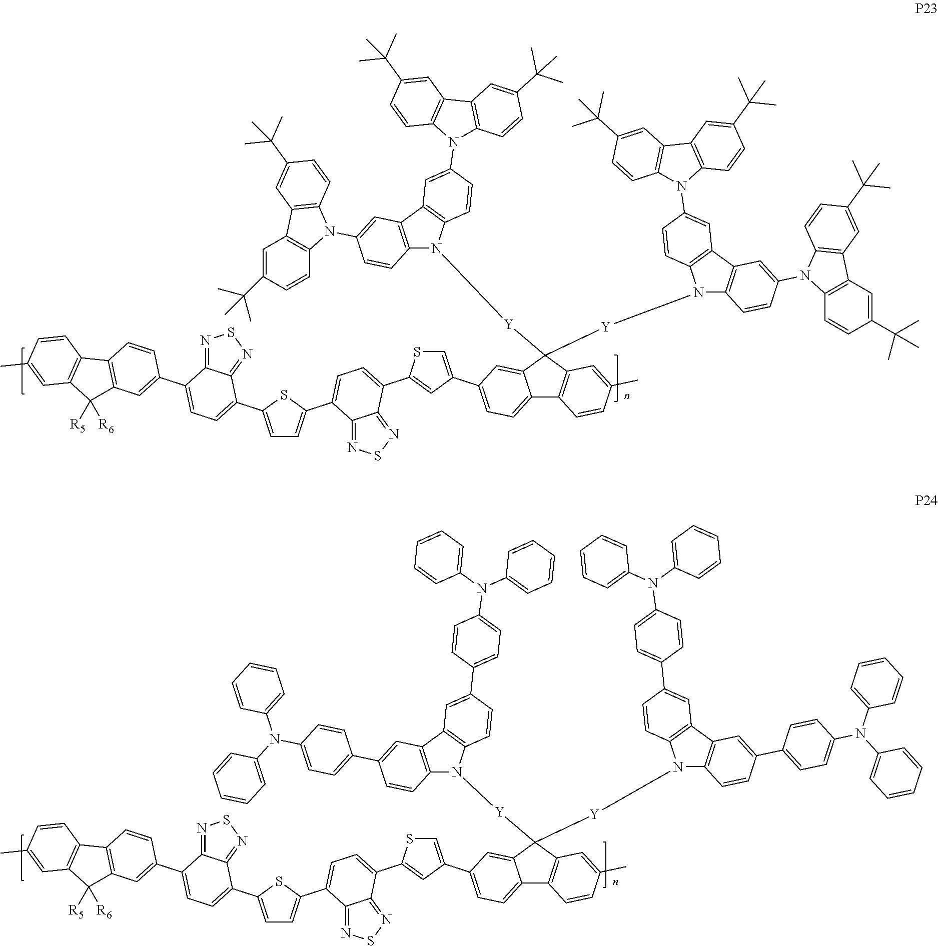

It is preferable that the white light-emitting polymer according to the present invention has a weight average molecular weight (Mw) in a range of 10,000 to 60,000 g/mol, a number average molecular weight (Mn) in a range of 10,000 to 36,000 g/mol and a polydispersity index (PDI) in a range of 1 to 5.

In a preferred embodiment, the white light-emitting polymer according to the present invention has a mole ratio of formula (II) to formula (I) to formula (III) to formula (IV) in a range of 1:0.3-1:0.002-0.5:0.008-0.7.

In a preferred embodiment, the white light-emitting polymer according to the present invention has a mole ratio of formula (II) to formula (I) to formula (III) to formula (V) in a range of 1:0.3-1:0.002-0.5:0.008-0.7.

In one embodiment, the white light-emitting polymer has a peak wavelength of light emitted in a visible spectrum of 360 nm to 380 nm.

In further embodiment, the white light-emitting polymer has CIE coordinates of light emission of x=0.27 to 0.35, y=0.20 to 0.33.

It is preferable that the polymer is used as a white electroluminescent material.

### Preparation of Polymer

In the present invention, the direct synthesis of small conjugated polymer nanoparticles (<30 nm) under mini-emulsion conditions via Glaser coupling reactions has been developed. The advantages of emulsion polymerization over the post-polymerization are scalable, which can obtain the concentration of polymer as 11 mg/ml, size controllable, and the uniform of particles. Due to the high stability of each droplet, evaporation of the solvent does not lead to any change of the droplet number. Therefore, using this mini-emulsion polymerization method in preparation of light-emitting conjugated polymer nanoparticles (NP) dispersion or light-emitting NP inks (EL inks) is attractive alternatives for commercial production of OLED devices by printing. The use of NPs dispersion in OLED fabrication promises of low temperature processing techniques and gives much lower viscosities than the equivalent loadings of high molar mass conjugated polymer dissolved in organic solvents.

The fourth aspect of the present invention relates to a method for preparing the polymers of the invention. The method comprises a polymerization of monomers comprising fluorene derivatives having structures of formulas (I) and (II) as described above.

In one embodiment, the monomers further comprise at least one compound having a structure of formulas (III), (IV) or (V) as described above.

In a preferred embodiment, the present invention provides a polymerization of monomers comprising the fluorene derivatives of formulas (I) and (II) and the compound of formula (III).

More preferably, the polymerization is conducted by using a mole ratio of the fluorene derivatives of formula (I):formula (II):the compound of formula (III) in a range of 0.3-1:1:0.005-0.5.

In one embodiment, the present invention provides a polymerization of monomers comprising the fluorene derivatives of formulas (I) and (II) and the compound of formula (IV).

More preferably, the polymerization is conducted by using a mole ratio of the fluorene derivatives of formula (I):formula (II):the compound of formula (IV) in a range of 0.3-1:1:0.008-0.7.

In one embodiment, the present invention provides a polymerization of monomers comprising the fluorene derivatives of formulas (I) and (II) and the compound of formula (V).

More preferably, the polymerization is conducted by using a mole ratio of the fluorene derivatives of formula (I):formula (II):the compound of formula (V) in a range of 0.3-1:1:0.008-0.7.

In one embodiment, the present invention provides a polymerization of monomers comprising the fluorene derivatives of formulas (I) and (II) and the compound of formulas (III) and (IV).

More preferably, the polymerization is conducted by using a mole ratio of the fluorene derivatives of formula (I):formula (II):the compound of formula (III):the compound of formula (IV) in a range of 0.3-1:1:0.002-0.5:0.008-0.7.

In one embodiment, the present invention provides a polymerization of monomers comprising the fluorene derivatives of formulas (I) and (II) and the compound of formulas (III) and (V).

More preferably, the polymerization is conducted by using a mole ratio of the fluorene derivatives of formula (I):formula (II):the compound of formula (III):the compound of formula (V) in a range of 0.3-1:1:0.002-0.5:0.008-0.7.

### Examples

The invention will now be described further by way of the following examples. The examples are for illustrative purposes and are not intended to limit the scope of the invention.

### 1. Preparation of Fluorene Derivatives

Examples of fluorene derivatives of formula (I) according to this invention are prepared by reacting the compound of formula (A) with the compound of formula (B). The compound (A) and (B) were prepared according to the following procedure.

### 1.1 Synthesis of Compound (A): 2,7-disubstituted-9,9-dihaloalkylfluorene

### Step 1: Synthesis of 2,7-dibromo-9H-fluorene

Fluorene (50 g) was dissolved in chloroform and then added FeCl₃ (0.3 g). After that, the solution of bromine 10% v/v chloroform was dropped at lower 10° C. and then the reaction mixture was stirred at room temperature for 3-5 hours. When the reaction completed, the reaction mixture was extracted with dichloromethane and brine solution. The resultant product was then dried over sodium sulfate (Na₂SO₄), concentrated on a rotary evaporator and recrystallized to give more than 70% yield of 2,7-dibromo-9H-fluorene.

### Step 2: Alkylation of 2,7-dibromo-9H-fluorene

2,7-dibromo-9H-fluorene (2 g) and tert-butylammonium bromide (0.1 g) were dissolved in 30 mL tetrahydrofuran and then the solution of KOH solution was added. The reaction mixture was stirred at room temperature for half an hour. After that, an alkylating agent (2 mL) was added and then stirred at 60-70° C. for 4-5 hours. The reaction was cooled to room temperature and the reaction mixture was poured in water, extracted with dichloromethane 3 times and brine solution. The resultant product was dried over sodium sulfate (Na₂SO₄) and concentrated on a rotary evaporator prior to purify by column chromatography to give 2,7-disubstituted-9,9-dihaloalkylfluorene.

The alkylating agents used in the step 2 above were varied resulting in different reaction products of the compound (A). Those different compounds A5-A8 obtained from using various alkylating agents are shown in Table 1 below.

TABLE 1

| Compound | Alkylating agent | Reaction Product |
|---|---|---|
| A5 | 1,3-dibromopropane | 2,7-dibromo-9,9-bis |
| | | (3-bromopropyl)-9H-fluorene |
| A6 | 1,6-dibromohexane | 2,7-dibromo-9,9-bis |
| | | (6-bromohexyl)-9H-fluorene |
| A7 | 1,8-dibromooctane | 2,7-dibromo-9,9-bis |
| | | (8-bromooctyl)-9H-fluorene |
| A8 | 1,10-dibromodecane | 2,7-dibromo-9,9-bis |
| | | (10-bromodecyl)-9H-fluorene |

### 1.2 Synthesis of Compound (B): 3,6-disubstituted 9H-carbazole

Examples of compound (B) obtained from the analogous manner with a variety of substituting groups are shown in Table 2 below.

TABLE 2

| Com- | Substituting | |
|---|---|---|
| pound | group | Reaction Product |
| B1 | tert-butyl | 3,6-di-tert-butyl-9H-carbazole |
| B2 | carbazole | 9′H-9,3′:6′,9″-tercarbazole |
| B3 | 3,6-di-tert-butyl- | 3,3″,6,6″-tetra-tert-butyl-9′H- |
| | carbazole | 9,3′:6′,9″-tercarbazole |
| B4 | TPA | 4,4′-(9H-carbazole-3,6- |
| | | diyl)bis(N,N-diphenylaniline) |

The synthesis of compounds B1-B4 are as described below.

### Synthesis of Compound B1: 3,6-di-tert-butyl-9H-carbazole

Carbazole (10 g) was dissolved in the mixed solvent of nitromethane and dichloromethane. ZnCl₂ (25 g) was added to the mixture solution, followed by dropwise addition of 2-chloro-2-methylpropane (20 mL) The reaction mixture was sonicated in an ultrasonic bath for at least 30 minutes. After that, the reaction mixture was poured into water, extracted with dichloromethane and brine solution. The resultant product was dried over sodium sulfate (Na₂SO₄) and concentrated on a rotary evaporator prior to purify by crystallization to give 3,6-di-tert-butyl-9H-carbazole.

### Synthesis of Compound B2: 9′H-9,3′:6′,9″-tercarbazole

—Ullmann Coupling Reaction of 3,6-diiodo-9-tosyl-9H-carbazole

3,6-diiodo-9-tosyl-9H-carbazole (1 g), carbazole (0.6 g), copper(I) iodide (0.1 g) and Potassium phosphate (K₃PO₄) (4.36 mmol) were dissolved in toluene. The mixture solution was added by trans-1,2-diaminocyclohexane (0.92 mmol), stirred at 110-120° C. and then refluxed for 40-50 hours. After the reaction completed, the reaction was cooled to room temperature and the reaction mixture was evaporated by using a rotary evaporator. Finally, the crude product was purified by crystallization with mixed solvent of dichloromethane and methanol.

—Deprotection of Tosyl Group

The deprotection of tosyl group was conducted by using potassium hydroxide as base and a mixture of tetrahydrofuran (THF) and dimethyl sulfoxide (DMSO) as solvent. The reaction mixture was added with small amount of water, stirred at 70-80° C. and then refluxed for 3-5 hours. After the reaction completed, the reaction mixture was poured in water, extracted with dichloromethane and brine solution. The resultant product was dried over sodium sulfate and concentrated by a rotary evaporator.

### Synthesis of Compound B3: 3,3″,6,6″-tetra-tert-butyl-9′H-9,3′:6′,9″-tercarbazole

—Ullmann Coupling Reaction of 3,6-diiodo-9-tosyl-9H-carbazole

3,6-diiodo-9-tosyl-9H-carbazole (1 g), 3,6-di-tert-butyl-9H-carbazole (0.6 g), copper(I) iodide (0.1 g) and Potassium phosphate (K₃PO₄) (4.36 mmol) were dissolved in toluene. The mixture solution was added by trans-1,2-diaminocyclohexane (0.92 mmol), stirred at 110-120° C. and then refluxed for 40-50 hours. After the reaction completed, the reaction was cooled to room temperature and the reaction mixture was evaporated by using a rotary evaporator. Finally, the crude product was purified by crystallization with mixed solvent of dichloromethane and methanol.

—Deprotection of Tosyl Group

The deprotection of tosyl group was conducted by using potassium hydroxide as base and a mixture of tetrahydrofuran (THF) and dimethyl sulfoxide (DMSO) as solvent. The reaction mixture was added with small amount of water, stirred at 70-80° C. and then refluxed for 3-5 hours. After the reaction completed, the reaction mixture was poured in water, extracted with dichloromethane and brine solution. The resultant product was dried over sodium sulfate and concentrated by a rotary evaporator.

### Synthesis of Compound B4: 4,4′-(9H-carbazole-3,6-diyl)bis(N,N-diphenylaniline)

3,6-diiodo-9H-carbazole (1 g) and 4-(Diphenylamino)phenylboronic acid (1.7 g) was dissolve in THF (30 mL). 2M Na₂CO₃ and Pd(PPh₃)₄ were added into the solution. The mixture was stirred and reflux at 75° C. for 18 h. After cool to room temperature, the mixture was poured in water and then extracted with dichloromethane (DCM, 3×50 mL) and brine solution (100 mL), dried over Na₂SO₄ and concentrated on a rotary evaporator. Finally, the residue was purified by column chromatography eluting with DCM/hexane to give white solid of 4,4′-(9H-carbazole-3,6-diyl)bis(N,N-diphenylaniline).

1.3 Synthesis of Fluorene Derivatives of the Invention

The fluorene derivatives of this invention were obtained by reacting the fluorene compounds A5-A8 with the carbazole compounds B1-B4. The synthesis follows the procedure as specified below.

The carbazole compound and potassium hydroxide were dissolved in dimethylformamide (DMF) and then added the fluorene compound. The reaction mixtures were stirred at room temperature for 4-5 hours. After the reaction completed, the reaction mixtures were poured in water and extracted with dichloromethane and brine solution. The resultant products were dried over sodium sulfate (Na₂SO₄) and evaporated by a rotary evaporator. Finally, the resultant products were purified by column chromatography to give fluorene derivatives

Various compounds of fluorene derivatives (I1-I16) obtained from the analogous manner using different fluorene compounds (A5-A8) and carbazole compounds (B1-B4) are shown in Table 3 below.

TABLE 3

| | | Fluorene derivative | |
|---|---|---|---|
| Fluorene | Carbazole | Example | |
| compound | compound | No. | Chemical name |
| A5 | B1 | I1 | 9,9′-((2,7-dibromo-9H-fluorene-9,9- |
| | | | diyl)bis(propane-3,1-diyl))bis(3,6-di-tert- |
| | | | butyl-9H-carbazole) |
| A6 | B1 | I2 | 9,9′-((2,7-dibromo-9H-fluorene-9,9-diyl)bis(hexane- |
| | | | 6,1-diyl))bis(3,6-di-tert-butyl-9H-carbazole) |
| A7 | B1 | I3 | 9,9′-((2,7-dibromo-9H-fluorene-9,9-diyl)bis(octane- |
| | | | 8,1-diyl))bis(3,6-di-tert-butyl-9H-carbazole) |
| A8 | B1 | I4 | 9,9′-((2,7-dibromo-9H-fluorene-9,9-diyl)bis(decane- |
| | | | 10,1-diyl))bis(3,6-di-tert-butyl-9H-carbazole) |
| A5 | B2 | I5 | 9′,9″″-((2,7-dibromo-9H-fluorene-9,9- |
| | | | diyl)bis(propane 3,1-diyl))bis(9′H-9,3′:6,9″- |
| | | | tercarbazole) |
| A6 | B2 | I6 | 9′,9″″-((2,7-dibromo-9H-fluorene-9,9- |
| | | | diyl)bis(hexane-6,1-diyl))bis(9′H-9,3′:6′,9″- |
| | | | tercarbazole) |
| A7 | B2 | I7 | 9′9″″-((2,7-dibromo-9H-fluorene-9,9- |
| | | | diyl)bis(octane-8,1-diyl))bis(9′H-9,3′:6′,9″- |
| | | | tercarbazole) |
| A8 | B2 | I8 | 9′9″″-((2,7-dibromo-9H-fluorene-9,9- |
| | | | diyl)bis(decane-10,1-diyl))bis(9′H-9,3′: 6′,9″- |
| | | | tercarbazole) |
| A5 | B3 | I9 | 9′,9″-((2,7-dibromo-9H-fluorene-9,9- |
| | | | diyl)bis(propane-3,1-diyl))bis(3,3″,6,6″-tetra-tert- |
| | | | butyl-9′H-9,3′:6,9″-tercarbazole) |
| A6 | B3 | I10 | 9′,9″″-((2,7-dibromo-9H-fluorene-9,9- |
| | | | diyl)bis(hexane-6,1-diyl))bis(3,3″,6,6″-tetra-tert- |
| | | | butyl-9′H-9,3′:6,9″-tercarbazole) |
| A7 | B3 | I11 | 9′,9″″-((2,7-dibromo-9H-fluorene-9,9- |
| | | | diyl)bis(octane-8,1-diyl))bis(3,3″,6,6″-tetra-tert- |
| | | | butyl-9′H-9,3′:6,9″-tercarbazole) |
| A8 | B3 | I12 | 9′,9″″-((2,7-dibromo-9H-fluorene-9,9- |
| | | | diyl)bis(decane-10,1-diyl))bis(3,3″,6,6″-tetra-tert- |
| | | | butyl-9′H-9,3′:6,9″-tercarbazole) |
| A5 | B4 | I13 | 4,4′,4″,4′″-(((2,7-dibromo-9H-fluorene-9,9- |
| | | | diyl)bis(propane-3,1-diyl))bis(9H-carbazole-9,3,6- |
| | | | triyl))tetrakis(N,N-diphenylaniline) |
| A6 | B4 | I14 | 4,4′4″,4″′-(((2,7-dibromo-9H-fluorene-9,9- |
| | | | diyl)bis(hexane-6,1-diyl))bis(9H-carbazole-9,3,6- |
| | | | triyl))tetrakis(N,N-diphenylaniline) |
| A7 | B4 | I15 | 4,4′4″,4″′-(((2,7-dibromo-9H-fluorene-9,9- |
| | | | diyl)bis(octane-8,1-diyl))bis(9H-carbazole-9,3,6- |
| | | | triyl))tetrakis(N,N-diphenylaniline) |
| A8 | B4 | I16 | 4,4′,4″,4″′-(((2,7-dibromo-9H-fluorene-9,9- |
| | | | diyl)bis(decane-10,1-diyl))bis(9H-carbazole-9, 3,6- |
| | | | triyl))tetrakis(N,N-diphenylaniline) |

The fluorene derivatives I1-I16 were characterized by ¹H NMR and Atmospheric pressure chemical ionization-Mass Spectrometry (APCI-MS) analysis. The exemplary characterization results for the fluorene derivatives I1-I4 of the present invention are shown below.

Compound I1

¹H NMR (600 MHz, CDCl₃): δ=8.03 (s, 4H), 7.46 (d, 2H, J=7.98 Hz), 7.43-7.40 (m, 6H), 7.26 (s, 4H), 6.94 (d, 4H, J=8.52 Hz), 3.88 (t, 4H, J=7.26, 7.32 Hz), 1.46 (s, 37H), 1.17-1.12 (m, 4H) ppm. APCI (m/z) calcd for C₆₅H₇₈Br₂N₂ [M⁺]: 962.3572; found 963.4061.

Compound I2

¹H NMR (600 MHz, CDCl₃): δ=8.07 (s, 4H), 7.48-7.47 (m, 6H), 7.43 (d, 2H, J=8.04 Hz), 7.38 (s, 2H), 7.21 (s, 2H), 7.19 (s, 2H), 4.11 (t, 4H J=7.02, 7.08 Hz), 1.85-1.83 (m, 4H), 1.66 (t, 4H, J=7.14, 7.14), 1.53 (s, 8H), 1.45 (s, 38H), 1.37-1.15 (m, 4H), 1.10-1.06 (m, 4H), 0.56-0.55 (m, 4H) ppm, APCI (m/z) calcd for C₆H₇₈Br₂N₂ [M⁺]:: 1046.4511; found 1047.5145.

Compound I3

¹H NMR (600 MHz, CDCl₃): δ=8.08 (s, 4H), 7.48-7.45 (m, 6H), 7.41 (d, 4H, J=7.5 Hz), 7.24 (s, 4H), 4.16 (t, 4H, J=6.96, 6.90 Hz), 1.89-1.86 (m, 4H), 1.76-1.74 (m, 4H), 1.45 (s, 36H), 1.29-1.25 (m, 5H), 1.12 (m, 4H), 1.01 (s, 8H), 0.55 (br. s, 4H), APCI (m/z) calcd for C₆₉H₈₆Br₂N₂ [M⁺]: 1102.5564; found 1103.5642.

Compound I4

¹H NMR (600 MHz, CDCl₃): δ=8.09 (br. d, 4H), 7.49 (br. d, 2H), 7.48 (br. d, 2H), 7.47-7.45 (m, 2H), 7.42-7.41 (m, 4H), 4.20 (t, 4H, J=6, 12 Hz), 1.90-1.88 (m, 4H), 1.83-1.73 (m, 4H), 1.46 (s, 36H) 1.36-1.30 (m, 4H), 1.15-1.11 (m, 4H), 1.06-1.02 (m, 12H) 0.56 (br. s, 4H) ppm.

1.4 Synthesis of Comparative Examples of Fluorene Derivatives

The comparative examples of fluorene derivatives were synthesized by the same procedure as specified in 1.3 using the fluorene compound A5-A8 but using 9H-carbazole as the carbazole compound. The obtained comparative fluorene derivatives are shown in Table 4 below.

TABLE 4

| Fluorene | Carbazole | Comparative fluorene derivative | |
|---|---|---|---|
| compound | compound | Example No. | Chemical name |
| A5 | 9H-carbazole | C1 | 9,9′-((2,7-dibromo-9H-fluorene- |
| | | | 9,9-diyl)bis(propane-3,1- |
| | | | diyl))bis(9H-carbazole) |
| A6 | 9H-carbazole | C2 | 9,9′-((2,7-dibromo-9H-fluorene- |
| | | | 9,9-diyl)bis(hexane-6,1- |
| | | | diyl))bis(9H-carbazole) |
| A7 | 9H-carbazole | C3 | 9,9′-((2,7-dibromo-9H-fluorene- |
| | | | 9,9-diyl)bis(octane-8,1- |
| | | | diyl))bis(9H-carbazole) |
| A8 | 9H-carbazole | C4 | 9,9′-((2,7-dibromo-9H-fluorene- |
| | | | 9,9-diyl)bis(decane-10,1- |
| | | | diyl))bis(9H-carbazole) |

The comparative fluorene compounds C₁-C₄ were characterized by ¹H NMR and Atmospheric pressure chemical ionization-Mass Spectrometry (APCI-MS) analysis. The characterization results for such comparative fluorene compounds are shown below.

Comparative C1

¹H-NMR (600 MHz, CDCl₃): δ=8.07 (d, 4H, J=12 Hz), 7.49-7.44 (m, 4H), 7.40 (t, 4H, J=12, 6 Hz), 7.29 (s, 2H), 7.20 (t, 4H, J=6, 6 Hz), 7.05 (d, 4H, J=12 Hz), 3.97 (t, 4H, J=12, 6 Hz), 2.00 (t, 4H, J=12, 6 Hz), 1.20-1.15 (m, 4H) ppm. APCI (m/z) calc for C₄₃H₃₄Br₂N₂ [M⁺]: 738.1068; found 739.1790.

Comparative C2

¹H-NMR (600 MHz, CDCl₃): δ=8.09-8.07 (m, 5H), 7.48 (d, 2H, J=12 Hz), 7.44-7.42 (m, 7H), 7.36 (s, 2H), 7.33-7.30 (m, 5H), 7.22-7.19 (m, 5H), 4.18 (t, 4H, J=6, 6 Hz), 1.82-1.80 (m, 5H), 1.72-1.67 (m, 4H), 1.16-1.06 (m, 8H), 0.53 (br. s, 4H) ppm. APCI (m/z) calc for C₄₉H₄₆Br₂N₂ [M⁺]: 822.2007; found 823.2857

Comparative C3

¹H-NMR (600 MHz, CDCl₃): δ=8.09 (d, 4H, J=6 Hz), 7.48-7.40 (m, 10H), 7.36 (d, 4H, J=12 Hz), 7.21 (t, 4H, J=6, 6 Hz), 4.24 (t, 4H, J=6, 6 Hz), 1.86 (t, 4H, J=6, 6 Hz), 1.81-1.76 (m, 4H), 1.86 (t, 4H, J=6, 6 Hz), 1.14-1.12 (m, 4H), 1.01 (s, 8H), 0.54 (brs, 4H) ppm. APCI (m/z) calc for C₅₃H₅₄Br₂N₂ [M⁺]: 878.2633; found 879.3067.

Comparative C4

¹H NMR (600 MHz, CDCl₃): δ=7.97 (d, 4H, J=6 Hz), 7.34-7.28 (m, 10H), 7.26 (d, 4H, J=12 Hz), 7.11-7.08 (m, 4H), 4.13 (t, 4H, J=12, 6 Hz), 1.76 (t, 4H, J=6, 12 Hz), 1.72-1.67 (m, 4H), 1.22-1.17 (m, 4H), 1.14-1.08 (m, 4H), 0.99-0.97 (m, 4H), 0.92-0.88 (m, 12H), 0.44 (br. s, 4H) ppm.

### 2. Preparation of Polymer

The polymers according to this invention were synthesized by mini-emulsion polymerization via Suzuki Miyaura cross-coupling of fluorene derivative monomers of the invention and at least one other monomer as described in detailed description of the invention. The examples and comparative examples were synthesized by following procedure.

The solution of tetraethylammonium hydroxide (Et₄NOH) (0.2 ml) was added to a three-neck flask containing 0.5 g of sodium dodecyl sulfate (SDS) and 50 mL of deionized water. The mixture solution was degassed by bubbling with nitrogen for 30-40 minutes. Monomers were dissolved in toluene, to which 100 uL of hexadecane was also added. The monomer solution was degassed by bubbling with nitrogen for 5-10 minutes. [1,1′-Bis(diphenylphosphino) ferrocene]palladium(II) dichloride (Pd(dppf)Cl₂) was added into the monomer solution, which then was transferred to the reaction vessel. The reaction mixture was emulsified by ultrasonication for 15-20 minutes while cooling with an ice bath. The three-neck flask was resealed, and the mini-emulsion polymerization was performed at 70-80° C. for 24-30 hours. After the polymerization completed, the reaction mixture was cooled to room temperature and precipitated in methanol. The reaction product was filtered and dried in an oven.

The resultant polymers obtained from the polymerization may be further purified to remove residue Pd catalyst which is the cause of dark spot in OLED device. The obtained polymers were extracted with Et₄NOH solution under reflux at 70-80° C. for 3-5 hours, two times.

In addition, the comparative polymers were obtained by the same procedure as specified in the method of polymerization but using the comparative fluorene derivatives C1-C4 instead of fluorene derivatives of the present invention.

The preparation of polymers with different color properties are specified below.

2.1 Blue Light-Emitting Polymer

Examples of blue light-emitting polymers are prepared from variations of substituents of the monomers as shown in Table 5 below.

TABLE 5

| | | | Functional | Mole ratio |
|---|---|---|---|---|
| | | | group of | of fluorene |
| | Functional group of fluorene | | fluorene | deriv- |
| | derivative of formula (I) | | derivative of | atives of |
| Example | R¹, R², R³ | n of Y group | formula (II) | formulas |
| No. | and R⁴ groups | (—CH₂)ₙ) | R⁵ and R⁶ group | (I):(II) |
| Compar- | H | 3 | C₈ alkyl | 1:1 |
| ative 1 | | | | |
| Compar- | H | 6 | C₈ alkyl | 1:1 |
| ative 2 | | | | |
| Compar- | H | 8 | C₈ alkyl | 1:1 |
| ative 3 | | | | |
| Compar- | H | 10 | C₈ alkyl | 1:1 |
| ative 4 | | | | |
| 1 | Tert-butyl | 3 | C₈ alkyl | 1:1 |
| 2 | Tert-butyl | 6 | C₈ alkyl | 1:1 |
| 3 | Tert-butyl | 8 | C₈ alkyl | 1:1 |
| 4 | Tert-butyl | 10 | C₈ alkyl | 1:1 |
| 5 | Carbazole | 3 | C₈ alkyl | 1:1 |
| 6 | Carbazole | 6 | C₈ alkyl | 1:1 |
| 7 | Carbazole | 8 | C₈ alkyl | 1:1 |
| 8 | Carbazole | 10 | C₈ alkyl | 1:1 |
| 9 | 3,6-di-tert-butyl | 3 | C₈ alkyl | 1:1 |
| | carbazole | | | |
| 10 | 3,6-di-tert-butyl | 6 | C₈ alkyl | 1:1 |
| | carbazole | | | |
| 11 | 3,6-di-tert-butyl | 8 | C₈ alkyl | 1:1 |
| | carbazole | | | |
| 12 | 3,6-di-tert-butyl | 10 | C₈ alkyl | 1:1 |
| | carbazole | | | |
| 13 | TPA | 3 | C₈ alkyl | 1:1 |
| 14 | TPA | 6 | C₈ alkyl | 1:1 |
| 15 | TPA | 8 | C₈ alkyl | 1:1 |
| 16 | TPA | 10 | C₈ alkyl | 1:1 |

2.2 Red Light-Emitting Polymer

Examples of red light-emitting polymers are prepared from variations of substituents of the monomers as shown in Table 6 below.

TABLE 6

| | | | Functional | Mole |
|---|---|---|---|---|
| | | | group | ratio of |
| | Functional group of fluorene | | of fluorene | fluorene |
| | derivative of formula (I) | | derivative of | derivatives |
| Example | R¹, R², R³ | n of Y group | formula (II) | of formulas |
| No. | and R⁴ | (—CH₂)ₙ) | R⁵ and R⁶ | (I):(II):(III) |
| Compar- | H | 3 | C₈ alkyl | 0.5:1:0.5 |
| ative 5 | | | | |
| Compar- | H | 6 | C₈ alkyl | 0.5:1:0.5 |
| ative 6 | | | | |
| Compar- | H | 8 | C₈ alkyl | 0.5:1:0.5 |
| ative 7 | | | | |
| Compar- | H | 10 | C₈ alkyl | 0.5:1:0.5 |
| ative 8 | | | | |
| 17 | Tert-butyl | 3 | C₈ alkyl | 0.5:1:0.5 |
| 18 | Tert-butyl | 6 | C₈ alkyl | 0.5:1:0.5 |
| 19 | Tert-butyl | 8 | C₈ alkyl | 0.5:1:0.5 |
| 20 | Tert-butyl | 10 | C₈ alkyl | 0.5:1:0.5 |
| 21 | Carbazole | 3 | C₈ alkyl | 0.5:1:0.5 |
| 22 | Carbazole | 6 | C₈ alkyl | 0.5:1:0.5 |
| 23 | Carbazole | 8 | C₈ alkyl | 0.5:1:0.5 |
| 24 | Carbazole | 10 | C₈ alkyl | 0.5:1:0.5 |
| 25 | 3,6-di-tert-butyl | 3 | C₈ alkyl | 0.5:1:0.5 |
| | carbazole | | | |
| 26 | 3,6-di-tert-butyl | 6 | C₈ alkyl | 0.5:1:0.5 |
| | carbazole | | | |
| 27 | 3,6-di-tert-butyl | 8 | C₈ alkyl | 0.5:1:0.5 |
| | carbazole | | | |
| 28 | 3,6-di-tert-butyl | 10 | C₈ alkyl | 0.5:1:0.5 |
| | carbazole | | | |
| 29 | TPA | 3 | C₈ alkyl | 0.5:1:0.5 |
| 30 | TPA | 6 | C₈ alkyl | 0.5:1:0.5 |
| 31 | TPA | 8 | C₈ alkyl | 0.5:1:0.5 |
| 32 | TPA | 10 | C₈ alkyl | 0.5:1:0.5 |

2.3 Green Light-Emitting Polymer

Examples of green light-emitting polymers are prepared from variations of substituents of the monomers as shown in Table 7 below.

TABLE 7

| | Functional | | Functional | Mole |
|---|---|---|---|---|
| | group of fluorene | | group | ratio of |
| | derivative of formula (I) | | of fluorene | Fluorene |
| | | n of Y | derivative of | derivative |
| | R¹, R², R³ | group | formula (II) | of formulas |
| Example No. | and R⁴ | (—CH₂)ₙ) | R⁵ and R⁶ | (I):(II):(V) |
| Comparative 9 | H | 3 | C₈ alkyl | 0.5:1:0.5 |
| Comparative 10 | H | 6 | C₈ alkyl | 0.5:1:0.5 |
| Comparative 11 | H | 8 | C₈ alkyl | 0.5:1:0.5 |
| Comparative 12 | H | 10 | C₈ alkyl | 0.5:1:0.5 |
| 33 | Tert-butyl | 3 | C₈ alkyl | 0.5:1:0.5 |
| 34 | Tert-butyl | 6 | C₈ alkyl | 0.5:1:0.5 |
| 35 | Tert-butyl | 8 | C₈ alkyl | 0.5:1:0.5 |
| 36 | Tert-butyl | 10 | C₈ alkyl | 0.5:1:0.5 |
| 37 | Carbazole | 3 | C₈ alkyl | 0.5:1:0.5 |
| 38 | Carbazole | 6 | C₈ alkyl | 0.5:1:0.5 |
| 39 | Carbazole | 8 | C₈ alkyl | 0.5:1:0.5 |
| 40 | Carbazole | 10 | C₈ alkyl | 0.5:1:0.5 |
| 41 | 3,6-di-tert-butyl | 3 | C₈ alkyl | 0.5:1:0.5 |
| | carbazole | | | |
| 42 | 3,6-di-tert-butyl | 6 | C₈ alkyl | 0.5:1:0.5 |
| | carbazole | | | |
| 43 | 3,6-di-tert-butyl | 8 | C₈ alkyl | 0.5:1:0.5 |
| | carbazole | | | |
| 44 | 3,6-di-tert-butyl | 10 | C₈ alkyl | 0.5:1:0.5 |
| | carbazole | | | |
| 45 | TPA | 3 | C₈ alkyl | 0.5:1:0.5 |
| 46 | TPA | 6 | C₈ alkyl | 0.5:1:0.5 |
| 47 | TPA | 8 | C₈ alkyl | 0.5:1:0.5 |
| 48 | TPA | 10 | C₈ alkyl | 0.5:1:0.5 |

2.4 White Light-Emitting Polymer

Examples of white light-emitting polymers are prepared from variations of substituents of the monomers as shown in Table 8 below.

TABLE 8

| | | | Functional | Mole |
|---|---|---|---|---|
| | Functional | | group | ratio of |
| | group of fluorene | | of fluorene | Fluorene |
| | derivative of formula (I) | | derivative of | derivative |
| Example | R¹, R², R³ | n of Y group | formula (II) | of formulas |
| No. | and R⁴ | (—CH₂)ₙ) | R⁵ and R⁶ | (I):(II):(III):(V) |
| Compar- | H | 3 | C₈ alkyl | 0.96:1:0.002:0.038 |
| ative 13 | | | | |
| Compar- | H | 6 | C₈ alkyl | 0.96:1:0.002:0.038 |
| ative 14 | | | | |
| Compar- | H | 8 | C₈ alkyl | 0.96:1:0.002:0.038 |
| ative 15 | | | | |
| Compar- | H | 10 | C₈ alkyl | 0.96:1:0.002:0.038 |
| ative 16 | | | | |
| 49 | Tert-butyl | 3 | C₈ alkyl | 0.96:1:0.002:0.038 |
| 50 | Tert-butyl | 6 | C₈ alkyl | 0.96:1:0.002:0.038 |
| 51 | Tert-butyl | 8 | C₈ alkyl | 0.96:1:0.002:0.038 |
| 52 | Tert-butyl | 10 | C₈ alkyl | 0.96:1:0.002:0.038 |
| 53 | Carbazole | 3 | C₈ alkyl | 0.96:1:0.002:0.038 |
| 54 | Carbazole | 6 | C₈ alkyl | 0.96:1:0.002:0.038 |
| 55 | Carbazole | 8 | C₈ alkyl | 0.96:1:0.002:0.038 |
| 56 | Carbazole | 10 | C₈ alkyl | 0.96:1:0.002:0.038 |
| 57 | 3,6-di-tert- | 3 | C₈ alkyl | 0.96:1:0.002:0.038 |
| | butyl carbazole | | | |
| 58 | 3,6-di-tert- | 6 | C₈ alkyl | 0.96:1:0.002:0.038 |
| | butyl carbazole | | | |
| 59 | 3,6-di-tert- | 8 | C₈ alkyl | 0.96:1:0.002:0.038 |
| | butyl carbazole | | | |
| 60 | 3,6-di-tert- | 10 | C₈ alkyl | 0.96:1:0.002:0.038 |
| | butyl carbazole | | | |
| 61 | TPA | 3 | C₈ alkyl | 0.96:1:0.002:0.038 |
| 62 | TPA | 6 | C₈ alkyl | 0.96:1:0.002:0.038 |
| 63 | TPA | 8 | C₈ alkyl | 0.96:1:0.002:0.038 |
| 64 | TPA | 10 | C₈ alkyl | 0.96:1:0.002:0.038 |

### 3. Determination of Characteristics of the Polymers

The examples of polymers from the above-described Tables 5-8 were further characterized by using various techniques.

3.1 Determination of Molecular Weight and Molecular Weight Distribution of Polymers

The weight average molecular weight (Mw), number average molecular weight (Mn) and polydispersity (PD) of the polymers were measured by Gel Permeation Chromatography (GPC), relative to a polystyrene standard.

5 mg of polymer example was dissolved in 2 mL of tetrahydrofuran (THF) at 35° C. for 45 minutes. X μL of the sample solution was injected into the GPC with RI detector (Malvern, United Kingdom) with flow rate of 1 mL/min at 35° C. in column zone and 35° C. in detector zone. The data was processed by Malvern Omnisec software, Malvern Viscotek TDAmax, country.

3.2 Determination of Shading and Optical Properties of Polymers

Color of the polymer was measured by using the coordinates of CIE 1931 XYZ color space chromaticity diagram.

Optical properties of the polymer were measured by Photoluminescence Spectroscopy and UV-Visible Spectroscopy in both diluted solution and film of samples.

UV-visible absorption of the polymer was detected by using Perkin-Elmer Lambda 1050 spectrometer.

Photoluminescence of the polymer was detected by using Edinburgh Instruments FLS 980 spectrometer. The samples were prepared by coating 2% w/v of the synthesized polymers in toluene on a glass substrate at 3000 rpm.

### 4. Results

4.1 Blue Light-Emitting Polymer

—Molecular Weight and Molecular Weight Distribution

The results of measurement of weight average molecular weight (Mw), number average molecular weight (Mn) and polydispersity (PDI) obtained by GPC technique are shown in Table 9 below.

TABLE 9

| Example | Mn (g/mol) | Mw (g/mol) | PDI |
|---|---|---|---|
| Comparative 1 | 8,721 | 18,315 | 2.100 |
| Comparative 2 | 21,611 | 45,615 | 2.111 |
| Comparative 3 | 20,121 | 33,278 | 1.654 |
| Comparative 4 | 19,057 | 39,039 | 2.049 |
| 1 | 11,045 | 23,794 | 2.154 |
| 2 | 4,258 | 6,086 | 1.429 |
| 3 | 29,233 | 56,937 | 1.938 |
| 4 | 19,750 | 41,769 | 2.115 |
| 5 | 11,092 | 20,293 | 1.829 |
| 6 | 11,995 | 26,818 | 2.236 |
| 7 | 19,899 | 36,461 | 1.832 |
| 8 | 13,190 | 20,938 | 1.587 |
| 9 | 9,331 | 12,554 | 1.344 |
| 10 | 12,547 | 18,872 | 1.504 |
| 11 | 11,703 | 16,904 | 1.444 |
| 12 | 13,533 | 18,310 | 1.353 |
| 13 | 9,259 | 14,657 | 1.583 |
| 14 | 16,932 | 30,846 | 1.822 |
| 15 | 9,393 | 11,916 | 1.268 |
| 16 | 13,562 | 22,607 | 1.667 |

—Optical Properties

The photoluminescence, UV-visible absorption and CIE coordinates results of obtained polymers from the polymerization according to the present are shown in Table 10 below.

TABLE 10

| | | Maximum | Maximum | |
|---|---|---|---|---|
| | | absorption | absorption | Photo- |
| | | peak | peak of | lumin- |
| | | of UV- | photo- | escence |
| | CIE | Visible | luminescence | quantum |
| Example | coordinates | spectrum | spectrum | yield |
| No. | (x, y) | (nm) | (nm) | (Φ, %) |
| Compar- | (0.17, 0.11) | 378 | 420 | 13 |
| ative 1 | | | | |
| Compar- | (0.17, 0.09) | 383 | 434 | 16 |
| ative 2 | | | | |
| Compar- | (0.17, 0.10) | 385 | 423 | 16 |
| ative 3 | | | | |
| Compar- | (0.17, 0.10) | 379 | 419 | 16 |
| ative 4 | | | | |
| 1 | (0.17, 0.10) | 377 | 417 | 22 |
| 2 | (0.18, 0.13) | 355 | 416 | 25 |
| 3 | (0.17, 0.07) | 384 | 417 | 16 |
| 4 | (0.17, 0.09) | 354 | 416 | 17 |
| 5 | (0.17, 0.09) | 374 | 423 | 15 |
| 6 | (0.16, 0.08) | 376 | 421 | 16 |
| 7 | (0.16, 0.08) | 383 | 422 | 21 |
| 8 | (0.17, 0.09) | 377 | 422 | 19 |

4.2 Red Light-Emitting Polymer

—Molecular Weight and Molecular Weight Distribution

The results of measurement of weight average molecular weight (Mw), number average molecular weight (Mn) and polydispersity (PDI) obtained by GPC technique are shown in Table 11 below.

TABLE 11

| Example | Mn (g/mol) | Mw (g/mol) | PDI |
|---|---|---|---|
| Comparative 5 | 21,400 | 28,900 | 1.35 |
| Comparative 6 | 21,957 | 29,345 | 1.34 |
| Comparative 7 | 23,876 | 29,877 | 1.25 |
| Comparative 8 | 23,368 | 28,497 | 1.22 |
| 17 | 18,344 | 31,400 | 1.71 |
| 18 | 19,456 | 31,675 | 1.62 |
| 19 | 19,432 | 30,966 | 1.594 |
| 20 | 20,554 | 31,868 | 1.55 |
| 21 | 22,600 | 32,899 | 1.45 |
| 22 | 25,744 | 35,233 | 1.37 |
| 23 | 26,877 | 33,856 | 1.25 |
| 24 | 27,655 | 35,899 | 1.29 |
| 25 | 23,987 | 33,125 | 1.38 |
| 26 | 24,777 | 32,658 | 1.32 |
| 27 | 28,976 | 31,455 | 1.08 |
| 28 | 26,588 | 32,635 | 1.23 |
| 29 | 24,567 | 33,555 | 1.36 |
| 30 | 26,678 | 31,699 | 1.18 |
| 31 | 26,788 | 35,899 | 1.34 |
| 32 | 29,743 | 35,134 | 1.18 |

—Optical Properties

The photoluminescence, UV-visible absorption and CIE coordinates results of obtained polymers from the polymerization according to the present are shown in Table 12 below.

TABLE 12

| | | Maximum | Maximum | |
|---|---|---|---|---|
| | | absorption | absorption | Photo- |
| | | peak | peak of | lumin- |
| | | of UV- | photo- | escence |
| | | Visible | luminescence | quantum |
| | CIE | spectrum | spectrum | yield |
| Example | coordinates | (nm) | (nm) | (Φ, %) |
| Compar- | (0.69, 0.30) | 380, 544 | 680 | 8.55 |
| ative 5 | | | | |
| Compar- | (0.69, 0.29) | 381, 545 | 677 | 9.64 |
| ative 6 | | | | |
| Compar- | (0.72, 0.31) | 382, 544 | 676 | 13.44 |
| ative 7 | | | | |
| Compar- | (0.70, 0.30) | 380, 545 | 680 | 14.78 |
| ative 8 | | | | |
| 17 | (0.71, 0.29) | 381, 550 | 678 | 15.66 |
| 18 | (0.72, 0.29) | 382, 551 | 678 | 15.66 |
| 19 | (0.70, 0.30) | 383, 546 | 686 | 19.31 |
| 20 | (0.71, 0.29) | 381, 550 | 678 | 15.66 |
| 21 | (0.70, 0.30) | 370, 546 | 674 | 9.55 |
| 22 | (0.72, 0.29) | 372, 551 | 680 | 14.73 |
| 23 | (0.68, 0.29) | 376, 545 | 677 | 13.55 |
| 24 | (0.70, 0.30) | 377, 550 | 675 | 14.51 |
| 25 | (0.71, 0.30) | 380, 548 | 687 | 16.78 |
| 26 | (0.70, 0.28) | 381, 546 | 675 | 16.22 |
| 27 | (0.70, 0.29) | 382, 549 | 685 | 22.56 |
| 28 | (0.72, 0.31) | 388, 550 | 680 | 15.46 |
| 29 | (0.71, 0.30) | 375, 543 | 677 | 11.12 |
| 30 | (0.70, 0.31) | 380, 550 | 680 | 15.44 |
| 31 | (0.70, 0.29) | 376, 544 | 670 | 14.56 |
| 32 | (0.72, 0.30) | 380, 552 | 669 | 16.78 |

4.3 Green Light-Emitting Polymer

—Molecular Weight and Molecular Weight Distribution

The results of measurement of weight average molecular weight (Mw), number average molecular weight (Mn) and polydispersity (PDI) obtained by GPC technique are shown in Table 13.

TABLE 13

| Example | Mn (g/mol) | Mw (g/mol) | PDI |
|---|---|---|---|
| Comparative 9 | 16,342 | 46,789 | 2.86 |
| Comparative 10 | 16,360 | 40,233 | 2.45 |
| Comparative 11 | 17,865 | 47,854 | 2.67 |
| Comparative 12 | 18,112 | 46,890 | 2.58 |
| 33 | 18,289 | 46,556 | 2.54 |
| 34 | 19,553 | 47,885 | 2.44 |
| 35 | 19,723 | 48,903 | 2.479 |
| 36 | 19,966 | 48,775 | 2.44 |
| 37 | 18,211 | 45,877 | 2.54 |
| 38 | 19,456 | 40,764 | 2.09 |
| 39 | 19,254 | 46,213 | 2.479 |
| 40 | 19,369 | 45,786 | 2.36 |
| 41 | 16,654 | 43,765 | 2.62 |
| 42 | 17,854 | 42,800 | 2.39 |
| 43 | 18,236 | 45,600 | 2.50 |
| 44 | 18,657 | 41,671 | 2.23 |
| 45 | 17,890 | 47,901 | 2.67 |
| 46 | 18,354 | 42,890 | 2.33 |
| 47 | 19,432 | 48,300 | 2.48 |
| 48 | 19,800 | 41,543 | 2.09 |

—Optical Properties

The photoluminescence, UV-visible absorption and CIE coordinates results of obtained polymers from the polymerization according to the present are shown in Table 14 below.

TABLE 14

| | | Maximum | Maximum | |
|---|---|---|---|---|
| | | absorption | absorption | Photo- |
| | | peak | peak of | lumin- |
| | | of UV- | photo- | escence |
| | | Visible | luminescence | quantum |
| | CIE | spectrum | spectrum | yield |
| Example | coordinates | (nm) | (nm) | (Φ, %) |
| Compar- | (0.34, 0.55) | 434 | 532 | 6.75 |
| ative 9 | | | | |
| Compar- | (0.33, 0.54) | 430 | 531 | 6.80 |
| ative 10 | | | | |
| Compar- | (0.36, 0.55) | 436 | 532 | 6.76 |
| ative 11 | | | | |
| Compar- | (0.36, 0.56) | 435 | 533 | 6.70 |
| ative 12 | | | | |
| 41 | (0.35, 0.52) | 435 | 533 | 10.78 |
| 42 | (0.36, 0.54) | 436 | 534 | 14.55 |
| 43 | (0.35, 0.60) | 430 | 535 | 16.89 |
| 44 | (0.33, 0.55) | 434 | 536 | 17.66 |
| 45 | (0.34, 0.55) | 437 | 535 | 10.88 |
| 46 | (0.34, 0.54) | 440 | 535 | 11.88 |
| 47 | (0.33, 0.58) | 432 | 532 | 14.55 |
| 48 | (0.35, 0.55) | 435 | 536 | 16.49 |

4.4 White Light-Emitting Polymer

—Molecular Weight and Molecular Weight Distribution

The results of measurement of weight average molecular weight (Mw), number average molecular weight (Mn) and polydispersity (PDI) obtained by GPC technique are shown in Table 15 below.

TABLE 15

| Example | Mn (g/mol) | Mw (g/mol) | PDI |
|---|---|---|---|
| Comparative 13 | 13,480 | 46,789 | 3.47 |
| Comparative 14 | 15,467 | 45,765 | 2.96 |
| Comparative 15 | 13,935 | 43,566 | 3.12 |
| Comparative 16 | 24,577 | 48,534 | 1.97 |
| 49 | 21,333 | 45,677 | 2.14 |
| 50 | 24,756 | 38,344 | 1.55 |
| 51 | 23,935 | 46,724 | 1.95 |
| 52 | 29,788 | 36,456 | 1.22 |
| 53 | 23,455 | 38,765 | 1.65 |
| 54 | 21,674 | 41,234 | 1.90 |
| 55 | 25,678 | 39,675 | 1.53 |
| 56 | 28,980 | 43,567 | 1.50 |
| 57 | 24,455 | 48,970 | 2.00 |
| 58 | 27,865 | 41,235 | 1.47 |
| 59 | 25,654 | 41,896 | 1.63 |
| 60 | 24,788 | 43,688 | 1.76 |
| 61 | 29,876 | 42,990 | 1.43 |
| 62 | 28,987 | 39,888 | 1.38 |
| 63 | 31,467 | 41,907 | 1.33 |
| 64 | 35,678 | 57,890 | 1.62 |

—Optical Properties

The photoluminescence, UV-visible absorption and CIE coordinates results of obtained polymers from the polymerization according to the present are shown in Table 16.

TABLE 16

| | | Maximum | Maximum |
|---|---|---|---|
| | | absorption | absorption peak |
| | | peak of UV- | of photo- |
| | CIE | Visible | luminescence |
| Example | coordinates | spectrum (nm) | spectrum (nm) |
| Compar- | (0.30, 0.31) | 367 | 445, 615 |
| ative 13 | | | |
| Compar- | (0.29, 0.28) | 370 | 451, 616 |
| ative 14 | | | |
| Compar- | (0.30, 0.31) | 371 | 450, 620 |
| ative 15 | | | |
| Compar- | (0.30, 0.28) | 372 | 451, 619 |
| ative 16 | | | |
| 49 | (0.33, 0.30) | 370 | 450, 620 |
| 50 | (0.28, 0.29) | 368 | 449, 618 |
| 51 | (0.32, 0.26) | 371 | 451, 618 |
| 52 | (0.28, 0.29) | 369 | 450, 617 |
| 53 | (0.33,0.20) | 372 | 451, 620 |
| 54 | (0.30, 0.28) | 370 | 451, 615 |
| 55 | (0.31,0.27) | 365 | 450, 620 |
| 56 | (0.33, 0.29) | 375 | 455, 619 |
| 57 | (0.35, 0.33) | 371 | 450, 622 |
| 58 | (0.31,0.33) | 370 | 451, 623 |
| 59 | (0.33, 0.30) | 371 | 450, 621 |
| 60 | (0.29, 0.30) | 370 | 451, 620 |
| 61 | (0.30, 0.31) | 369 | 450, 615 |
| 62 | (0.27, 0.30) | 371 | 451, 620 |
| 63 | (0.28, 0.30) | 367 | 450, 617 |
| 64 | (0.31,0.33) | 371 | 445, 615 |

The synthesized white light-emitting polymers were further measured photoluminescence quantum yield (Φ) to compare with the comparative examples 13-16. From the experimental results, it shows that the obtained polymers of the present invention provide higher photoluminescence quantum yields than that of the comparative examples when compared with the same number of carbon atoms of —(CH₂)ₙ— group of the fluorene derivatives of formula (I). For example, when n is 3 the photoluminescence quantum yield of the comparative example 13 is 20% while that of white polymer of the present invention is 30% (example 57) and when n is 8 the photoluminescence quantum yield of the comparative example 15 is 31% while that of the white polymers of the present invention are 48% (examples 51) and 40% (example 59).

1. A fluorene derivative having a structure of formula (I): 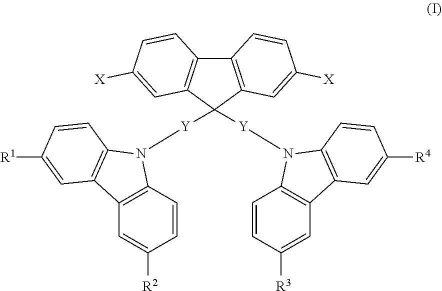
wherein
X is independently selected from a group consisting of Cl, Br, I, trifluoromethanesulfonate
and 4-(trifluoromethyl)benzenesulfonate;
Y is a group of formula —(CH₂)ₙ— where n is an integer ranging from 3 to 12; and
R¹, R², R³ and R⁴ are independently selected from a group consisting of tert-butyl group, triphenylamine, and tert-butyl-substituted carbazole.

2-3. (canceled)

4. The fluorene derivative according to claim 1, wherein X is Br or I, Y is a group of formula —(CH₂)ₙ— where n is an integer ranging from 3 to 10, and R¹, R², R³ and R⁴ are identical.

5. A method for preparing a fluorene derivative having a structure of formula (I): 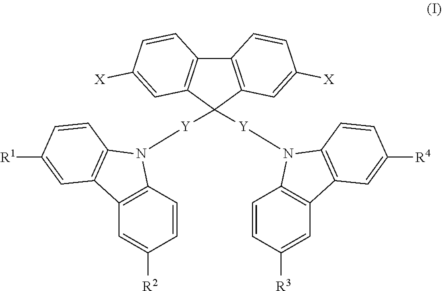
the method comprising reacting a compound of formula (A) with a compound of formula (B) in an organic solvent in the presence of a strong base, 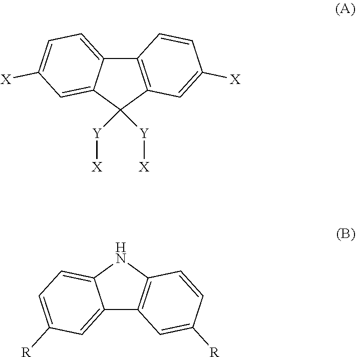
wherein
X is independently selected from a group consisting of Cl, Br, I, trifluoromethanesulfonate and 4-(trifluoromethyl)benzenesulfonate;
Y is a group of formula —(CH₂)ₙ— where n is an integer ranging from 3 to 12;
R¹, R², R³, R⁴, and R are independently selected from a group consisting of tert-butyl group, triphenylamine, and tert-butyl-substituted carbazole; and
wherein a mole ratio of the compound of formula (A) to the compound of formula (B) to the strong base ((A):(B):strong base) is in a range of 1:2.5-3:8-9.

6. (canceled)

7. The method according to claim 5, wherein the compound of formula (A) is prepared from steps comprising:
reacting a fluorene compound with a halogenating agent or triflating agent in an organic solvent in the presence of a catalyst; and
(ii) reacting the compound obtained from the step (i) with a C₃-C₁₂ alkyl halide in an organic solvent in the presence of the halogenating agent or triflating agent and a strong base wherein the step (i) is conducted at an ambient temperature for 3-5 hours and the step (ii) is conducted at a temperature of 70-80° C. for 4-5 hours;
wherein a mole ratio of the fluorene compound to the halogenating agent or triflating agent to the catalyst in step (i) (fluorene compound:halogenating agent or triflating agent:catalyst) is in a range of 1:2-3:0.05-0.1, and a mole ratio of the compound obtained from step (i) to the C₃-C₁₂ alkyl halide to the halogenating agent or triflating agent to the strong base (compound obtained from step (i): alkyl halide:halogenating agent or triflating agent:strong base) is in a range of 1:3-5:0.1-0.3:8-15; and
wherein the halogenating agent is dibromine (Br₂) or tert-butyl ammonium bromide, the triflating agent is trifluoromethanesulfonate or 4-(trifluoromethyl) benzenesulfonate, the C₃-C₁₂ alkyl halide is selected from a group consisting of 1,3-dibromopropane, 1,6-dibromohexane, 1,8-dibromooctane and 1,10-dibromodecane, and the catalyst is metal halide.

8-14. (canceled)

15. The method according to claim 5, wherein the compound of formula (B) is prepared by reacting carbazole with a halogenating agent or triflating agent in a mixed organic solvent in the presence of a catalyst, and a mole ratio of carbazole to the halogenating agent or triflating agent to the catalyst (carbazole:halogenating agent or triflating agent:catalyst) is in a range of 1:2.5-3.5:2.5-3.5, preferably the halogenating agent is 2-chloro-2-methylpropane, the catalyst is metal halide, and the mixed organic solvent is a mixture of nitromethane and dichloromethane and a volume ratio of nitromethane and dichloromethane is 2 to 1.

16-19. (canceled)

20. The method according to claim 5, wherein the compound of formula (B) is prepared by steps of:
(i) reacting 3,6-dihalo-N-tosyl carbazole with carbazole or carbazole derivative in an organic solvent in the presence of a catalyst and a strong base; and
(ii) deprotecting a tosyl group of the compound obtained from step (i) in a mixed organic solvent in the presence of a strong base
wherein the step (i) is conducted at a temperature of 110-120° C. under refluxing condition for 40-50 hours, the catalyst of step (i) is prepared from metal halide and diamine compound, a mole ratio of 3,6-dihalo-N-tosyl carbazole to carbazole or carbazole derivative to the catalyst (3,6-dihalo-N-tosyl carbazole:carbazole or carbazole derivative:the catalyst) is in a range of 1:2.2-2.5:0.5-0.8; and
wherein the step (ii) is conducted at a temperature of 70-80° C. under refluxing condition for 3-5 hours, and the mixed organic solvent of step (ii) is a mixture of tetrahydrofuran and dimethyl sulfoxide.

21-25. (canceled)

26. A polymer comprising a repeating unit of a fluorene derivative having a structure of formula (I): 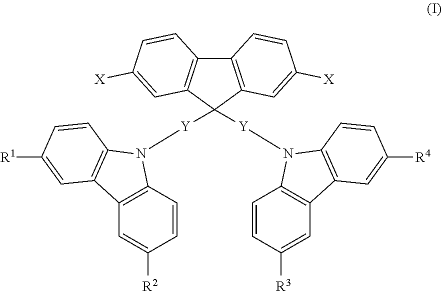
wherein
X is independently selected from a group consisting of Cl, Br, I, trifluoromethanesulfonate and 4-(trifluoromethyl)benzenesulfonate;
Y is a group of formula —(CH₂)ₙ— where n is an integer ranging from 3 to 12; and
R¹, R², R³ and R⁴ are independently selected from a group consisting of tert-butyl group, triphenylamine (TPA), and tert-butyl-substituted carbazole.

27. The polymer according to claim 26, wherein R¹, R², R³ and R⁴ are identical X is Br or I, and Y is a group of formula —(CH₂)ₙ— where n is an integer ranging from 3 to 10.

28-29. (canceled)

30. The polymer according to claim 26, comprising a repeating unit of fluorene derivatives of formulas (I) and (II): 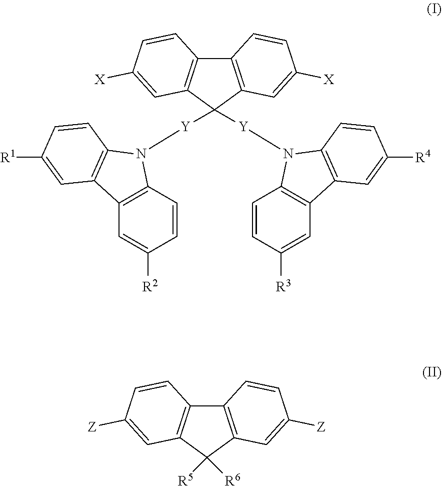
wherein
X is independently selected from a group consisting of Cl, Br, I, trifluoromethanesulfonate and 4-(trifluoromethyl)benzenesulfonate;
Y is a group of formula —(CH₂)ₙ— where n is an integer ranging from 3 to 12;
R¹, R², R³, and R^{4,} are independently selected from a group consisting of tert-butyl group, triphenylamine, and tert-butyl-substituted carbazole;
Z is dioxaborolane; and
R⁵ and R⁶ are independently selected from C₃-C₁₂ alkyl group.

31. The polymer according to claim 30, wherein Y is a group of formula —(CH₂)ₙ— where n is an integer ranging from 3 to 10, R¹, R², R³, and R⁴ are identical, and R⁵ and R⁶ are identical and are C₆-C₁₀ alkyl group.

32-34. (canceled)

35. The polymer according to claim 30, having a weight average molecular weight (Mw) in a range of 6,000 to 60,000 g/mol, a number average molecular weight (Mn) in a range of 4,200 to 30,000 g/mol, and a polydispersity index (PDI) in a range of 1 to 5.

36. The polymer according to claim 30, having a mole ratio of formula (II) to formula (I) in a range of 1:0.3-1, preferably 1 to 1.

37-40. (canceled)

41. The polymer according to claim 30, having a peak wavelength of light emitted in a visible spectrum of 350 nm to 385 nm, and CIE coordinates of light emission of x=0.16 to 0.18, y=0.07 to 0.13.

42. The polymer according to claim 30 which is used as a blue electroluminescent material.

43. The polymer according to claim 26, comprising a repeating unit of fluorene derivatives of formulas (I) and (II) and a compound of formula (III): 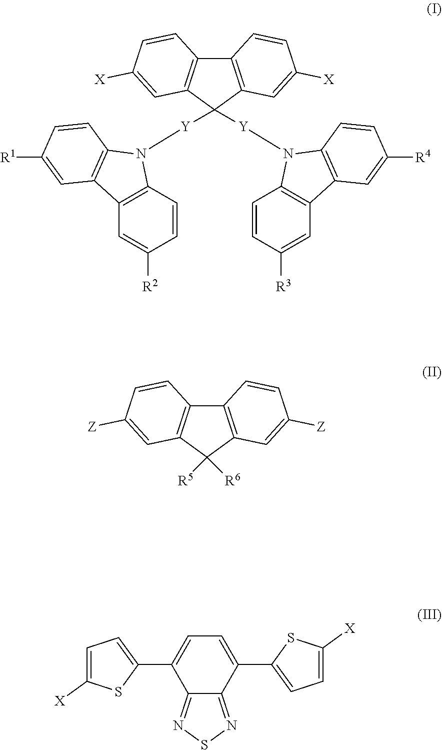
wherein
X is independently selected from a group consisting of Cl, Br, I, trifluoromethanesulfonate and 4-(trifluoromethyl)benzenesulfonate;
Y is a group of formula —(CH₂)ₙ— where n is an integer ranging from 3 to 12;
R¹, R², R³, and R^{4,} are independently selected from a group consisting of tert-butyl group, triphenylamine, and tert-butyl-substituted carbazole;
Z is dioxaborolane; and
R⁵ and R⁶ are independently selected from C₃-C₁₂ alkyl group.

44. The polymer according to claim 43, wherein Y is a group of formula —(CH₂)ₙ— where n is an integer ranging from 3 to 10, R¹, R², R³, and R⁴ are identical, and R⁵ and R⁶ are identical and are C₆-C₁₀ alkyl group.

45-47. (canceled)

48. The polymer according claim 43, having a weight average molecular weight (Mw) in a range of 10,000 to 100,000 g/mol, a number average molecular weight (Mn) in a range of 10,000 to 35,000 g/mol, and a polydispersity index (PDI) in a range of 1 to 5.

49-50. (canceled)

51. The polymer according to claim 43, having a mole ratio of formula (II) to formula (I) to formula (III) in a range of 1:0.3-1:0.005-0.5.

52. The polymer according to claim 43, having a peak wavelength of light emitted in a visible spectrum of 370 to 390 nm and 540 nm to 560 nm, and CIE coordinates of light emission of x=0.68 to 0.72, y=0.28 to 0.31.

53. (canceled)

54. The polymer according to claim 43, which is used as a red electroluminescent material.

55. The polymer according to claim 26, comprising a repeating unit of fluorene derivatives of formulas (I) and (II) and a compound of formulas (IV) or (V): 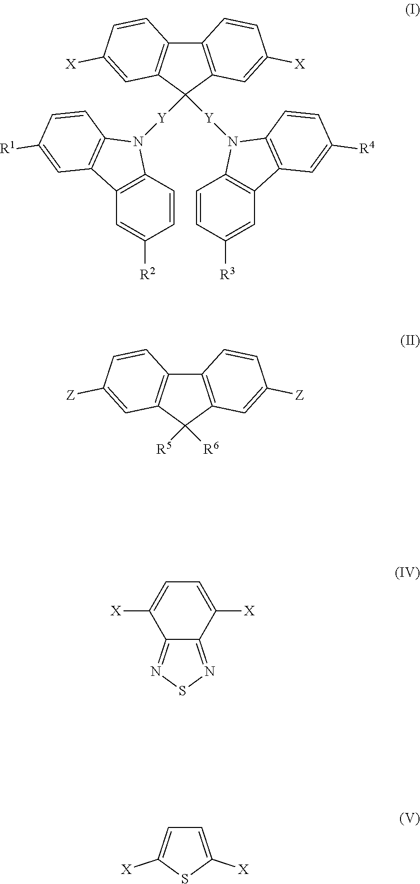
wherein
X is independently selected from a group consisting of Cl, Br, I, trifluoromethanesulfonate and 4-(trifluoromethyl)benzenesulfonate;
Y is a group of formula —(CH₂)ₙ— where n is an integer ranging from 3 to 12;
R¹, R², R³, and R^{4,} are independently selected from a group consisting of tert-butyl group, triphenylamine, and tert-butyl-substituted carbazole;
Z is dioxaborolane; and
R⁵ and R⁶ are independently selected from C₃-C₁₂ alkyl group.

56. The polymer according to claim 55, wherein Y is a group of formula —(CH₂)ₙ— where n is an integer ranging from 3 to 10, R¹, R², R³ and R⁴ are identical, and R⁵ and R⁶ are identical and are C₆-C₁₀ alkyl group.

57-59. (canceled)

60. The polymer according to claim 55, having a weight average molecular weight (Mw) in a range of 10,000 to 50,000 g/mol, a number average molecular weight (Mn) in a range of 10,000 to 30,000 g/mol, and a polydispersity index (PDI) in a range of 1 to 5.

61-62. (canceled)

63. The polymer according to claim 55, having a mole ratio of formula (II) to formula (I) to formula (IV) or (V) in a range of 1:0.3-1:0.008-0.7.

64. (canceled)

65. The polymer according to claim 55, having a peak wavelength of light emitted in a visible spectrum of 430 nm to 440 nm, and CIE coordinates of light emission of x=0.32 to 0.36, y=0.52 to 0.60.

66. (canceled)

67. The polymer according to claim 55, which is used as a green electroluminescent material.

68. The polymer according to claim 26, comprising a repeating unit of fluorene derivatives of formulas (I) and (II) and a compound of formula (III) and a compound of formulas (IV) or (V): 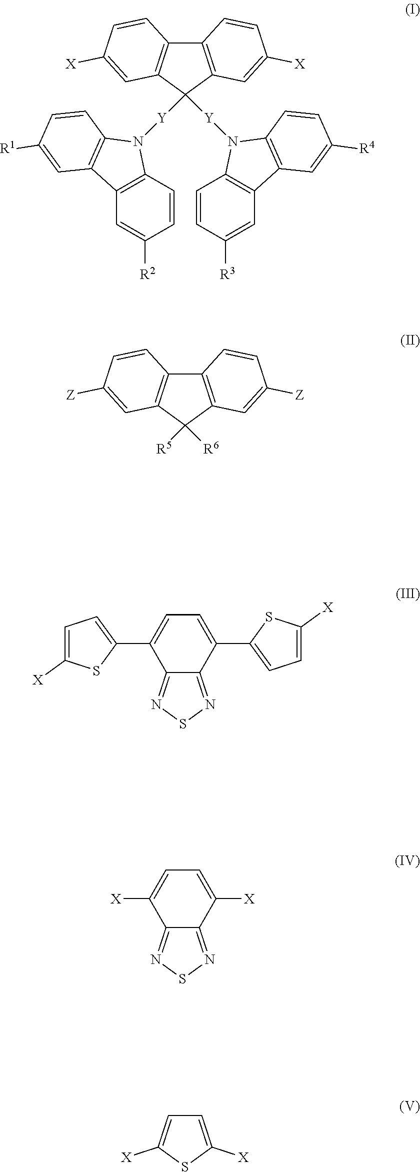
wherein
X is independently selected from a group consisting of Cl, Br, I, trifluoromethanesulfonate and 4-(trifluoromethyl)benzenesulfonate;
Y is a group of formula —(CH₂)ₙ— where n is an integer ranging from 3 to 12;
R¹, R², R³, and R^{4,} are independently selected from a group consisting of tert-butyl group, triphenylamine, and tert-butyl-substituted carbazole;
Z is dioxaborolane; and
R⁵ and R⁶ are independently C₃-C₁₂ alkyl group.

69. The polymer according to claim 68, wherein Y is a group of formula —(CH₂)ₙ— where n is an integer ranging from 3 to 10, R¹, R², R³ and R⁴ are identical, and R⁵ and R⁶ are identical and are C₆-C₁₀ alkyl group.

70-72. (canceled)

73. The polymer according to claim 68, having a weight average molecular weight (Mw) in a range of 10,000 to 60,000 g/mol, a number average molecular weight (Mn) in a range of 10,000 to 36,000 g/mol, and a polydispersity index (PDI) in a range of 1 to 5.

74-75. (canceled)

76. The polymer according to claim 68, having a mole ratio of formula (II) to formula (I) to formula (III) to formula (IV) or (V) in a range of 1:0.3-1:0.002-0.5:0.008-0.7.

77. (canceled)

78. The polymer according to claim 68, having a peak wavelength of light emitted in a visible spectrum of 360 nm to 380 nm, and CIE coordinates of light emission of x=0.27 to 0.35, y=0.20 to 0.33.

79. (canceled)

80. The polymer according to claim 68, which is used as a white electroluminescent material.

81. The polymer according to claim 26 which is used as a hole transport layer in electroluminescent device.

82. A light-emitting element comprising the polymer according to claim 26.

83. A method for preparing the polymer according to claim 26, comprising a polymerization of monomers comprising fluorene derivatives having structures of formulas (I) and (II): 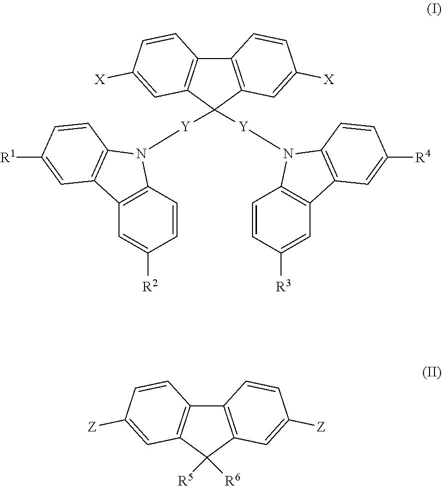
wherein
X is independently selected from a group consisting of Cl, Br, I, trifluoromethanesulfonate and 4-(trifluoromethyl)benzenesulfonate;
Y is a group of formula —(CH₂)ₙ— where n is an integer ranging from 3 to 12;
R¹, R², R³, and R^{4,} are independently selected from a group consisting of tert-butyl group, triphenylamine, and tert-butyl-substituted carbazole;
Z is dioxaborolane; and
R⁵ and R⁶ are independently selected from C₃-C₁₂ alkyl group.

84. (canceled)

85. The method according to claim 83, wherein a mole ratio of the fluorene derivatives of formula (I) to formula (II) is in a range of 0.3-1:1.

86. The method according to claim 83, wherein the polymerization is conducted at a temperature of 70-80° C.

87. The method according to claim 83, wherein the monomers further comprising at least one compound having a structure of formulas (III), (VI) or (V): 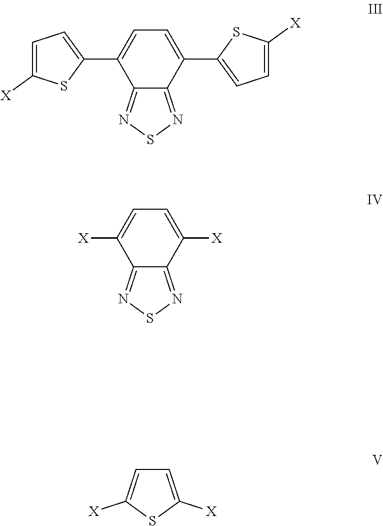
wherein
X is independently selected from a group consisting of Cl, Br, I, trifluoromethanesulfonate and 4-(trifluoromethyl)benzenesulfonate.

88. The method according to claim 87, wherein a mole ratio of the fluorene derivatives of formula (I) to formula (II) to the compound of formula (III) is in a range of 0.3-1:1:0.005-0.5.

89. The method according to claim 87, wherein a mole ratio of the fluorene derivatives of formula (I) to formula (II) to the compound of formula (IV) or (V) is in a range of 0.3-1:1:0.008-0.7.

90. (canceled)

91. The method according to claim 87, wherein a mole ratio of the fluorene derivatives of formula (I) to formula (II) to the compound of formula (III) to the compound of formula (IV) or (V) is in a range of 0.3-1:1:0.002-0.5:0.008-0.7.

92. (canceled)
